(19) United States
(12) Patent Application Publication
(10) Pub. No.: US 2024/0052407 A1
(43) Pub. Date: Feb. 15, 2024

(54) TERTIARY COOPERATIVE PRIMERS AND METHODS OF USE

(71) Applicant: Co-Diagnostics, Inc., Salt Lake City, UT (US)

(72) Inventors: Mark Aaron Poritz, Salt Lake City, UT (US); Kirk Max Ririe, Salt Lake City, UT (US); Madison Blair Edwards, Cottonwood Heights, UT (US); Jana Kent, Salt Lake City, UT (US); Ali S. Sharif, West Valley City, UT (US)

(21) Appl. No.: 18/192,914

(22) Filed: Mar. 30, 2023

Related U.S. Application Data
(60) Provisional application No. 63/325,851, filed on Mar. 31, 2022.

(51) Int. Cl. C12Q 1/6853

(52) U.S. Cl. CPC C12Q 1/6853; C12Q 1/686

(57) ABSTRACT

Specifically, disclosed herein is a tertiary cooperative nucleic acid sequence. This sequence can comprise a first extendable nucleic acid sequence configured to hybridize to a first region of a target nucleic acid; a second nucleic acid sequence that comprises at least one non-extendable moiety at it's 3′ terminus, wherein said non-extendable moiety renders said second nucleic acid sequence non-extendable, wherein said second nucleic acid sequence is located on a 5′ side of the first nucleic acid on the molecule and is configured to hybridize to a second region of the target nucleic acid downstream from a binding site of the first nucleic acid sequence relative to its direction of extension; a first spacer separating the first and second nucleic acid sequences configured to allow both sequences to hybridize to their targets, and comprising at least one non-nucleotide linker; a third nucleic acid sequence that comprises at least one non-extendable moiety at it's 3′ terminus, wherein said non-extendable moiety renders said third nucleic acid sequence non-extendable, wherein said third nucleic acid sequence is located on a 5′ side of the second nucleic acid on the molecule and is configured to hybridize to a third region of the target nucleic acid between where the 3′ end of the first nucleic acid sequence hybridizes and where the 5′ end of the second nucleic acid sequence hybridizes. Also disclosed are methods and kits using the tertiary cooperative nucleic acid sequence.

### CROSS-REFERENCE TO RELATED APPLICATIONS

This application claims benefit of U.S. Provisional Application No. 63/325,851, filed Mar. 31, 2022, incorporated herein by reference in its entirety.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Aug. 30, 2023, is entitled “10393-015WO1-US.xml”, and is 69,481 bytes in size

### FIELD OF THE INVENTION

The disclosed invention is generally in the field of nucleic acid amplification.

### BACKGROUND OF THE INVENTION

Nucleic acid testing often requires amplification of nucleic acids to achieve a sufficient concentration and/or purity to undergo subsequent testing. Sometimes amplification of nucleic acids is used as a surrogate in detection of non-nucleic acids, such as proteins. The majority of nucleic acid amplification/extension reactions depend on the presence of a primer comprised of modified or natural nucleic acids at the 3′ end which allow extension in the presence of a polymerase.

A universal problem with such amplification reactions is the presence of primer-dimers as well as non-specific amplification. Primer-dimers are formed when primers extend each other rather than the target nucleic acid. Primer-dimers use up primers, resulting in the presence of impurities in the reaction. Even worse, primer-dimers can use up enough primers to cause false negatives in some cases. Or, if interacting with a probe, primer-dimers can cause false positives. Since nonspecific amplification products can be generated during assembly of PCR reactions, a method is needed that can reduce or eliminate these artifacts. Various methods have been developed to address this problem. These techniques are generally known as “hot-start” methods because the primer extension reactions are not allowed to “start” until stringent or “hot” hybridization temperatures have been reached.

Various hot start methods include suspending the polymerase in a wax material, inhibiting the polymerase with antibodies, chemically modifying the polymerase, sequestering primers, and a variety of other methods. The problem with all of these methods is that they are only effective prior to the first round of amplification/extension. Any primer-dimers that form thereafter are amplified at an exponential rate. Other methods of dealing with primer-dimers include methods such as nested PCR.

What is needed in the art are primers and methods of using them which improve the specificity and speed of nucleic acid amplification. It is further needed that such primers work with short discontinuous consensus regions for hybridization as may be the case with highly polymorphic genes and genome sequence targets.

### BRIEF SUMMARY OF THE INVENTION

Specifically, disclosed herein is a tertiary cooperative nucleic acid molecule. This molecule can comprise a first extendable nucleic acid sequence configured to hybridize to a first region of a target nucleic acid; a second nucleic acid sequence that is non-extendable, wherein said second nucleic acid sequence is located on a 5′ side of the first nucleic acid on the molecule and is configured to hybridize to a second region of the target nucleic acid downstream from a binding site of the first nucleic acid sequence relative to its direction of extension; a first spacer connecting the first and second nucleic acid sequences configured to allow both sequences to hybridize to their targets, and comprising at least one non-nucleotide linker, wherein the non-nucleotide linker causes the extension of a complementary strand to terminate; a third nucleic acid sequence that is non-extendable, wherein said third nucleic acid sequence is located on a 5′ side of the second nucleic acid on the molecule and is configured to hybridize to a third region of the target nucleic acid between where the 3′ end of the first nucleic acid sequence hybridizes and where the 5′ end of the second nucleic acid sequence hybridizes; a second spacer connecting the second and third nucleic acid sequences optionally comprising a non-nucleotide linker. Also disclosed are methods, reaction mixtures, and kits using the tertiary cooperative nucleic acid sequence.

### BRIEF DESCRIPTION OF THE DRAWINGS

FIG. 1A-C shows the general structure of a tertiary cooperative primer (CopTer), and how it and a traditional cooperative primer may each hybridize to their target nucleic acids. FIG. 1A shows that a CopTer has an anchor sequence 1 at its 5′ terminal designed to hybridize to a region on a target sequence, followed by a spacer 2, followed by a second anchor sequence 3 designed to hybridize to another region on the target, followed by a second spacer 4 and a priming sequence 5 with its direction of extension shown by the arrowhead. FIG. 1B shows a CopTer binding to its target sequence in which two anchor sequences (1 and 3) bind downstream to the binding site of the priming sequence 5, and the resulting CopTer-target complex having two nonhybridized loops on the target (6 and 7). FIG. 1C shows a traditional cooperative primer binding to its target sequence in which a single anchor sequence 1 is bound downstream of the priming sequence 5 resulting in one nonhybridized loop on the target 6.

FIG. 2A-C shows example configurations of tertiary cooperative primers (CopTers) for amplification. FIG. 2A shows CopTer pairs interdigitated to generate a short amplicon. The forward CopTer (1a-5a) hybridizes to template strand 21, and the reverse CopTer (1b-5b) hybridizes to template strand 20. FIG. 2B shows forward and reverse CopTer pairs spaced apart to allow room for a probe 9 to hybridize between binding sites of anchors 3a and 3b on the resulting amplicon. FIG. 2C shows one CopTer paired with a non-CopTer primer 8 to generate an amplicon, optionally allowing space for probe 9 to hybridize.

FIG. 3A-B shows amplification data from the study described in Example 1. FIG. 3A shows a temperature profile, and FIG. 3B shows real-time amplification curves generated by a CopTer and standard (non-cooperative) primer pair. Duplicate samples containing template generated robust amplification (solid lines, FIG. 3B) while duplicate samples of no template controls (dashed lines, FIG. 3B) showed no amplification.

FIG. 4A-F shows amplification data. FIG. 4A-C shows amplification curves generated from the study described in Example 2 in which the annealing temperature during amplification was 55° C., and FIG. 4D-F shows derivative melting curves observed after amplification. FIGS. 4A and 4D are results generated by use of standard (non-cooperative) primer pairs, FIGS. 4B and 4E by cooperative primer pairs, and FIGS. 4C and 4F by CopTer pairs. Shown are samples containing template (solid lines), no template controls (dashed lines), and samples without reverse transcriptase (alternating line and dashes).

FIG. 5A-F shows amplification data. FIG. 5A-C shows curves from the study in Example 2 in which the annealing temperature during amplification was 60° C., and FIG. 5D-F shows derivative melting curves observed after amplification. FIGS. 5A and 5D are results generated by standard (non-cooperative) primer pairs, FIGS. 5B and 5E by cooperative primer pairs, and FIGS. 5C and 5F by CopTer pairs. Shown are samples containing template (solid lines), no template controls (dashed lines), and samples without reverse transcriptase (alternating line and dashes).

FIG. 6A-F shows amplification data. FIG. 6A-C shows amplification curves from the study in Example 2 in which the annealing temperature during amplification was 67° C., and FIG. 6D-F shows derivative melting curves observed after amplification. FIGS. 6A and 6D are results generated by standard (non-cooperative) primer pairs, FIGS. 6B and 6E by cooperative primer pairs, and FIGS. 6C and 6F by CopTer pairs. Shown are samples containing template (solid lines), no template controls (dashed lines), and samples without reverse transcriptase (alternating line and dashes).

FIG. 7A-D shows amplification data. FIG. 7A-B shows amplification curves from the study in Example 3 in which the annealing temperature during amplification was 67° C., and FIG. 7C-D shows derivative melting curves observed after amplification. FIGS. 7A and 7C are results generated by standard (non-cooperative) primer pairs, and FIGS. 7B and 7D by CopTer pairs. Shown are samples containing template (solid lines), and no template controls (dashed lines).

FIG. 8A-D shows amplification data from the study in Example 4. FIG. 8A-B shows amplification curves in which the annealing temperature during amplification was 67° C., and FIG. 8C-D shows derivative melting curves observed after amplification. FIGS. 8A and 8C are results generated by CopTer primer pairs directed to a SARS-CoV-2 segment starting at 981 nt, and FIGS. 8B and 8D by CopTer primer pairs directed to SARS-CoV-2 segment starting at 12539 nt. Shown are samples containing template (solid lines), no template controls (dashed lines), and samples without reverse transcriptase (alternating line and dashes).

### DETAILED DESCRIPTION OF THE INVENTION

The disclosed method makes use of certain materials and procedures which allow amplification of nucleic acid sequences and whole genomes or other highly complex nucleic acid samples. These materials and procedures are described in detail below.

### Definitions

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used herein, “nucleic acid sequence” refers to the order or sequence of nucleotides along a strand of nucleic acids. In some cases, the order of these nucleotides may determine the order of the amino acids along a corresponding polypeptide chain. The nucleic acid sequence thus codes for the amino acid sequence. The nucleic acid sequence may be single-stranded or double-stranded, as specified, or contain portions of both double-stranded and single-stranded sequences. The nucleic acid sequence may be composed of DNA, both genomic and cDNA, RNA, or a hybrid, where the sequence comprises any combination of deoxyribo- and ribo-nucleotides, and any combination of bases, including uracil (U), adenine (A), thymine (T), cytosine (C), guanine (G), inosine, xathanine hypoxathanine, isocytosine, isoguanine, etc. It may include modified bases, including locked nucleic acids, peptide nucleic acids and others known to those skilled in the art.

The term “nucleoside”, as used herein, refers to a compound comprising a nucleotide base linked to the C-1′ carbon of a sugar, such as ribose, arabinose, xylose, and pyranose, and sugar analogs thereof. The sugar may be substituted or unsubstituted. Substituted ribose sugars include, but are not limited to, those riboses in which one or more of the carbon atoms, for example the 2′-carbon atom, is substituted with one or more of the same or different, —R, —OR, —NR2 azide, cyanide or halogen groups, where each R is independently H, C1-C6 alkyl, C2-C7 acyl, or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT Published Application Nos. WO 98/22489, WO 98/39352, and WO 99/14226; and Braasch and Corey, Chem. Biol. 8:1-7, 2001; and U.S. Pat. No. 6,268,490).

“LNA” or “locked nucleic acid” is a nucleotide analog that is conformationally locked such that the ribose ring is constrained by a methylene linkage between, for example but not limited to, the 2′-oxygen and the 3′- or 4′-carbon or a 3′-4′ LNA with a 2′-5′ backbone (see, e.g., Imanishi and Obika, U.S. Pat. No. 6,268,490; and Wengel and Nielsen, U.S. Pat. No. 6,670,461). The conformation restriction imposed by the linkage often increases binding affinity for complementary sequences and increases the thermal stability of such duplexes.

The 2′- or 3′-position of ribose can be modified to include hydrogen, hydroxy, methoxy, ethoxy, allyloxy, isopropoxy, butoxy, isobutoxy, methoxyethyl, alkoxy, phenoxy, azido, cyano, amido, imido, amino, alkylamino, fluoro, chloro and bromo. Nucleotides include the natural D optical isomer, as well as the L optical isomer forms (see, e.g., Garbesi et al., Nucl. Acids Res. 21:4159-65 (1993); Fujimori et al., J. Amer. Chem. Soc. 112:7436-38 (1990); Urata et al., Nucl. Acids Symposium Ser. No. 29:69-70 (1993)). When the nucleotide base is a purine, e.g., A or G, the ribose sugar is attached to the N9-position of the nucleotide base. When the nucleotide base is a pyrimidine, e.g. C, T, or U, the pentose sugar is attached to the N1-position of the nucleotide base, except for pseudouridines, in which the pentose sugar is attached to the C5 position of the uracil nucleotide base (see, e.g., Kornberg and Baker, DNA Replication, 2nd Ed. (1992), Freeman, San Francisco, Calif.).

The term “nucleotide 5′-triphosphate” refers to a nucleotide with a triphosphate ester group at the 5′ position, and is sometimes denoted as “rNTP”, or “dNTP” and “ddNTP” to particularly point out the structural features of the ribose sugar, or generically as “NTP”. The triphosphate ester group may include sulfur substitutions for the various oxygens, e.g., α-thio-nucleotide 5′-triphosphates. Reviews of nucleotide chemistry can be found in, among other places, Miller, Bioconjugate Chem. 1:187-91 (1990); Shabarova, Z. and Bogdanov, A. Advanced Organic Chemistry of Nucleic Acids, VCH, New York (1994); and Nucleic Acids in Chemistry and Biology, 2d ed., Blackburn and Gait, eds., Oxford University Press (1996; hereinafter “Blackburn and Gait”).

The term “nucleotide analogs” refers to synthetic analogs having modified nucleotide base portions, modified pentose portions, and/or modified phosphate portions, and, in the case of polynucleotides, modified internucleotide linkages, as generally described herein and elsewhere (e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Generally, modified phosphate portions comprise analogs of phosphate wherein the phosphorous atom is in the +5 oxidation state and one or more of the oxygen atoms is replaced with a non-oxygen moiety, for example but not limited to, sulfur. Some non-limiting examples of phosphate analogs include phosphorothioate, phosphorodithioate, phosphoroselenoate, phosphorodiselenoate, phosphoroanilothioate, phosphoranilidate, phosphoramidate, boronophosphates, including associated counterions, e.g., H+, NH4+, Na+, if such counterions are present. Non-limiting examples of modified nucleotide base portions include 5-methylcytosine (5mC); C-5-propynyl analogs, including but not limited to, C-5 propynyl-C and C-5 propynyl-U; 2,6-diaminopurine, also known as 2-amino adenine or 2-amino-dA; hypoxanthine, pseudouridine, 2-thiopyrimidine, isocytosine (isoC), 5-methyl isoC, and isoguanine (isoG; see, e.g., U.S. Pat. No. 5,432,272). Non-limiting examples of modified pentose portions include LNA analogs including without limitation Bz-A-LNA, 5-Me-Bz-C-LNA, dmf-G-LNA, and T-LNA (see, e.g., The Glen Report, 16(2):5 (2003); Koshkin et al., Tetrahedron 54:3607-30 (1998)), and 2′- or 3′-modifications where the 2′- or 3′-position is hydrogen, hydroxy, alkoxy (e.g., methoxy, ethoxy, allyloxy, isopropoxy, butoxy, isobutoxy and phenoxy), azido, amino, alkylamino, fluoro, chloro, or bromo. Modified internucleotide linkages include phosphate analogs, analogs having achiral and uncharged intersubunit linkages (e.g., Sterchak, E. P. et al., Organic Chem. 52:4202 (1987)), and uncharged morpholino-based polymers having achiral intersubunit linkages (see, e.g., U.S. Pat. No. 5,034,506). Some non-limiting examples of internucleotide linkage analogs include morpholidate, acetal, and polyamide-linked heterocycles. In one class of nucleotide analogs, known as peptide nucleic acids, including without limitation pseudocomplementary peptide nucleic acids (collectively “PNA”), a conventional sugar and internucleotide linkage has been replaced with a 2-aminoethylglycine amide backbone polymer (see, e.g., Nielsen et al., Science, 254:1497-1500 (1991); Egholm et al., J. Am. Chem. Soc., 114: 1895-1897 (1992); Demidov et al., Proc. Natl. Acad. Sci. 99:5953-58 (2002); Peptide Nucleic Acids: Protocols and Applications, Nielsen, ed., Horizon Bioscience (2004)). A wide range of nucleotide analogs for use in enzymatic incorporation or chemical synthesis are available as triphosphates, phosphoramidates, or CPG derivatives from, among other sources, Glen Research, Sterling, Md.; Link Technologies, Lanarkshire, Scotland, UK; and TriLink BioTechnologies, San Diego, Calif. Descriptions of oligonucleotide synthesis and certain nucleotide analogs, can be found in, among other places, S. Verma and F. Eckstein, Ann. Rev. Biochem. 67:99-134 (1999); Goodchild, Bioconj. Chem. 1:165-87 (1990); Current Protocols in Nucleic Acid Chemistry, Beaucage et al., eds., John Wiley & Sons, New York, N.Y., including updates through August 2005 (hereinafter “Beaucage et al.”); and Blackburn and Gait.

As used herein, the term “detecting” used in context of detecting a signal from a detectable label to indicate the presence of a target nucleic acid in the sample does not require the method to provide 100% sensitivity and/or 100% specificity. As is well known, “sensitivity” is the probability that a test is positive, given that the sample has a target nucleic acid sequence, while “specificity” is the probability that a test is negative, given that the sample does not have the target nucleic acid sequence. A sensitivity of at least 50% is preferred, although sensitivities of at least 60%, at least 70%, at least 80%, at least 90% and at least 99% are clearly more preferred. A specificity of at least 50% is preferred, although sensitivities of at least 60%, at least 70%, at least 80%, at least 90% and at least 99% are clearly more preferred. Detecting also encompasses assays with false positives and false negatives. False negative rates may be 1%, 5%, 10%, 15%, 20% or even higher. False positive rates may be 1%, 5%, 10%, 15%, 20% or even higher. The term “detecting” is also used in the context of detecting the amplified target nucleic acid by its melting temperature using melting curve analysis, as is known in the art.

A “fragment” in the context of a nucleic acid refers to a sequence of contiguous nucleotide residues which are at least about 5 nucleotides, at least about 7 nucleotides, at least about 9 nucleotides, at least about 111 nucleotides, or at least about 17 nucleotides. The fragment is typically less than about 300 nucleotides, less than about 100 nucleotides, less than about 75 nucleotides, less than about 50 nucleotides, or less than 30 nucleotides. In certain embodiments, the fragments can be used in polymerase chain reaction (PCR) or various hybridization procedures to identify or amplify identical or related parts of mRNA or DNA molecules. A fragment or segment may uniquely identify each polynucleotide sequence of the present invention.

As used herein, “labels” are chemical or biochemical moieties useful for labeling a nucleic acid (including a single nucleotide), amino acid, or antibody. “Labels” include fluorescent agents, chemiluminescent agents, chromogenic agents, quenching agents, radionuclides, enzymes, substrates, cofactors, inhibitors, magnetic particles, quantum dots, and other moieties known in the art. “Labels” are capable of generating a measurable signal, or can be used to capture nucleic acids, and may be covalently or noncovalently joined to an oligonucleotide or nucleotide (e.g., a non-natural nucleotide).

The term “multiplex PCR” as used herein refers to an assay that provides for simultaneous amplification of two or more products within the same reaction vessel. Each product is primed using a primer pair. A multiplex reaction may further include labeled primers each product, that are detectably labeled with different detectable moieties.

An “oligonucleotide” is a polymer comprising two or more nucleotides. The polymer can additionally comprise non-nucleotide elements such as labels, quenchers, blocking groups, or the like. The nucleotides of the oligonucleotide can be natural or non-natural and can be unsubstituted, unmodified, substituted or modified. The nucleotides can be linked by phosphodiester bonds, or by phosphorothioate linkages, methylphosphonate linkages, boranophosphate linkages, or the like.

A “peptide nucleic acid” (PNA) is a polymer comprising two or more peptide nucleic acid monomers. The polymer can additionally comprise elements such as labels, quenchers, blocking groups, or the like. The monomers of the PNA can be unsubstituted, unmodified, substituted or modified.

A “primer” is a nucleic acid that contains a sequence complementary to a region of a template nucleic acid strand and that primes the synthesis of a strand complementary to the template (or a portion thereof). Primers are typically, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified. By “normal primer” is meant a primer which does not have one or more anchor sequences, or second or third nucleic acid sequences, attached to it via a spacer. Normal primers are also referred to herein as “standard” or “non-cooperative” primers).

“Downstream” is relative to the action of the polymerase during nucleic acid synthesis or extension. For example, when the Taq polymerase extends a primer, it adds bases to the 3′ end of the primer and will move towards a sequence that is “downstream from the 3′ end of the primer.”

A “target region” is a region of a target nucleic acid that is to be amplified, detected or both.

The “Tm” (melting temperature) of a nucleic acid duplex under specified conditions is the temperature at which half of the nucleic acid sequences are disassociated and half are associated. As used herein, “isolated Tm” refers to the individual melting temperature of either the first or second nucleic acid sequence in the cooperative nucleic acid when not in the cooperative pair. “Effective Tm” refers to the resulting melting temperature of either the first or second nucleic acid when linked together.

The term “spacer” means the composition joining the first and second nucleic acids to each other, or the second and third nucleic acids to each other (or, if more than three nucleic acid sequences that hybridize with the target exist in the tertiary cooperative primer, the spacer can be between a third and fourth nucleic acid, or a fifth and sixth, etc). The spacer can comprise at least one non-extendable moiety, but may also comprise extendable nucleic acids, and can be any length. The spacer may be connected to the 3′ end, the 5′ end, or can be connected one or more bases from the end (“the middle”) of both the first and second nucleic acid sequences. By “end” is meant the terminal nucleic acid sequence. The connection can be covalent, hydrogen bonding, ionic interactions, hydrophobic interactions, and the like. The term “non-extendable” has reference to the inability of DNA polymerase to recognize a moiety and thereby continue nucleic acid synthesis. A variety of natural and modified nucleic acid bases are recognized by the polymerase and are “extendable.” Examples of non-extendable moieties include among others, fluorophores, quenchers, polyethylene glycol, polypropylene glycol, polyethylene, polypropylene, polyamides, polyesters, 3′ blockers, and others known to those skilled in the art. These are described in more detail below. In some cases, even a nucleic acid base with reverse orientation (e.g. 5′ ACGT 3′ 3′A 5′ 5′ AAGT 3′) or otherwise rendered such that the polymerase could not extend through it could be considered “non-extendable.”

The term “non-nucleic acid linker” or “non-nucleotide linker” as used interchangeably herein refers to a reactive chemical group that is capable of covalently attaching a first nucleic acid to a second nucleic acid, or more specifically, the primer to the anchor sequences. Suitable flexible linkers are typically linear molecules in a chain of at least one or two atoms, more typically an organic polymer chain of 1 to 12 carbon atoms (and/or other backbone atoms) in length. Exemplary flexible linkers include polyethylene glycol, polypropylene glycol, polyethylene, polypropylene, polyamides, polyesters and the like. The non-nucleic acid linkers can be non-extendable. Examples include, but are not limited to, C3, C6, and C12 spacers, 18-atom non-nucleotide hexa-ethyleneglycol, 9-atom triethylene glycol PEG-150, and abasic tetrahydrofuran (dSpacer).

As used herein, “complementary” or “complementarity” refers to the ability of a nucleotide in a polynucleotide molecule to form a base pair with another nucleotide in a second polynucleotide molecule. For example, the sequence 5′-A-C-T-3′ is complementary to the sequence 3′-T-G-A-5′. Complementarity may be partial, in which only some of the nucleotides match according to base pairing, or complete, where all the nucleotides match according to base pairing. For purposes of the present invention “substantially complementary” refers to 90% or greater identity over the length of the target base pair region. The complementarity can also be 50, 60, 70, 75, 80, 85, 90, 91, 92, 93, 94, 95, 96, 97, 98, 99, or 100% complementary, or any amount below or in between these amounts.

The terms “amplification” or “amplify” as used herein includes methods for copying a target nucleic acid, thereby increasing the number of copies of a selected nucleic acid sequence. Amplification may be exponential or linear. A target nucleic acid may be either DNA or RNA. The sequences amplified in this manner form an “amplicon” or “amplification product.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam, et al., Nucleic Acids Res. 2001 Jun. 1; 29(11):E54-E54; Hafner, et al., Biotechniques 2001 30(4):852-6, 858, 860; Zhong, et al., Biotechniques 2001 30(4):852-6, 858, 860.

The terms “target nucleic acid” or “target sequence” as used herein refer to a sequence which includes a segment of nucleotides of interest to be amplified and detected. Copies of the target sequence which are generated during the amplification reaction are referred to as amplification products, amplimers, or amplicons. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion or duplication, tandem repeat regions, a gene of interest, a region of a gene of interest or any upstream or downstream region thereof. Target nucleic acids may represent alternative sequences or alleles of a particular gene. Target nucleic acids may be derived from genomic DNA, cDNA, or RNA. As used herein target nucleic acid may be DNA or RNA extracted from a cell or a nucleic acid copied or amplified therefrom.

As used herein, “purified” refers to a polynucleotide, for example a target nucleic acid sequence, that has been separated from cellular debris, for example, high molecular weight DNA, RNA and protein. This would include an isolated RNA sample that would be separated from cellular debris, including DNA. It can also mean non-native, or non-naturally occurring nucleic acid.

As used herein, “protein,” “peptide,” and “polypeptide” are used interchangeably to denote an amino acid polymer or a set of two or more interacting or bound amino acid polymers.

As used herein, “stringency” refers to the conditions, i.e., temperature, ionic strength, solvents, and the like, under which hybridization between polynucleotides occurs. Hybridization being the process that occurs between the primer and template DNA during the annealing step of the amplification process.

A variety of additional terms are defined or otherwise characterized herein. “Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are specifically incorporated by reference. Nothing herein is to be construed as an admission that the present invention is not entitled to antedate such disclosure by virtue of prior invention.

Those skilled in the art will recognize, or be able to ascertain using no more than routine experimentation, many equivalents to the specific embodiments of the method and compositions described herein. Such equivalents are intended to be encompassed by the following claims.

### Tertiary Cooperative Primers and Methods of Use

The present invention provides methods that are generally applicable to the detection of nucleic acids, and particularly well-suited to the detection of target genomes or gene regions that are highly variable. The methods are also well-suited to shortening the time of nucleic acid amplification as they permit the use of high-temperature annealing conditions and narrow temperature ranges for temperature cycling.

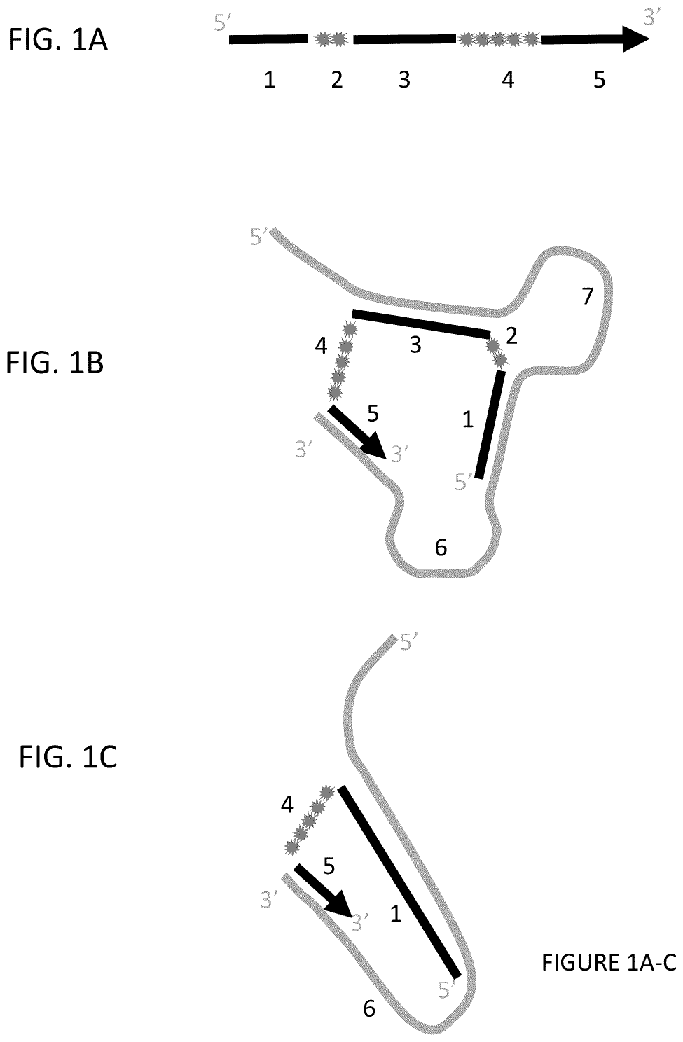

In particular, the invention provides methods that improve the specificity of nucleic acid amplification by use of a tertiary cooperative primer (CopTer) schematically shown in FIG. 1A. A CopTer comprises a short extendable priming sequence (labeled 5) that is covalently linked to two non-extendable anchor sequences 1 and 3 where each of the three are designed to hybridize to unique complementary sequences on the same target strand (FIG. 1B). Priming sequence 5 is linked to anchor sequence 3 through a spacer 4 comprising a non-nucleotide linker, while the two anchor sequences 1 and 3 are linked through another spacer 2 which may, or may not, include a non-nucleotide linker. Both anchors are designed to hybridize downstream to where the priming sequence hybridizes (downstream relative to the direction of extension) and to create unhybridized single-stranded gaps or loops 6 and 7 on the target-CopTer complex. During amplification (and during reverse transcription, if applicable) anchors 1 and 3 are either digested, or displaced (resulting in a single-stranded tail) depending on the type of polymerase used. In contrast, a traditional cooperative primer as described by Satterfield (U.S. Pat. Nos. 10,093,966, 10,704,087) and schematically shown in FIG. 1C uses only one anchor 1 to stabilize the target-cooperative primer complex.

Much like traditional PCR primer designs, CopTers require use of conserved sequences in the target organism for successful amplification. However, when contiguous stretches of conserved sequences are hard to find, CopTers allow the use of a short priming sequence that is stabilized by two discontinuous anchor sequences. Stabilization of the CopTer-target hybrid can be optimized by varying the length of the gaps between priming and anchor sequences, or between the two anchors, as well as the lengths and placement of the anchor sequences. This ability to fine-tune primer performance for amplification using not one, but two anchors, and two loops/gaps is an improvement over previous cooperative primers.

Another advantage of CopTers is the ability to perform PCR using high annealing temperatures to ensure high specificity of amplification and fast cycling time. Traditional hot-start techniques provide specificity by decreasing unwanted non-specific priming in the first cycle of amplification, while the use of a CopTer provides specificity during all cycles of amplification. To achieve this effect, a CopTer must have (a) an extendable priming sequence with a low melting temperature (Tm) as possible, between 40 and 60° C., preferably between 45 and 55° C., and (b) two anchor sequences with Tms higher than the priming sequence, between 55 and 72° C., preferably between 62 and 70° C. When all three sequences (priming and two anchor sequences) are hybridized to their complementary targets, the effective Tm of the priming sequence is increased by at least 1° C. The priming sequence must be long enough for DNA polymerase to successfully interact with the primer-target duplex, i.e. at least eight base pairs according to the crystal structure of Taq polymerase bound to the 3′ end of such duplex (Eom et al, 1996). Thus, to achieve the desired low Tm, the priming sequence preferably is between 8 and 16 bases, more preferably between 10 and 14 bases. Longer priming sequences also can be used as intended if its GC content is low, or if the PCR buffer contains melting-temperature depressors, non-limiting examples of which include DMSO, glycerol, betaine, formamide, and 1,2-propanediol. Temperature cycling for amplification with CopTers is performed between the denaturing temperature (typically 88-95° C.) and the high annealing temperature (for example 70° C.), resulting in faster cycling time due to the narrow temperature range used. The low Tm of the priming sequence of the CopTer helps mitigate any annealing or extension by itself on a non-target sequence due to the absence of stabilization by cooperative anchors hybridized to their targets. With one or two anchor sequences annealed to their targets, however, the CopTer-target duplex can be designed to have a very high Tm without influencing the Tm of the amplicon. If a non-CopTer primer is designed to act as part of a primer pair with a CopTer, such primer will need to function at the same high annealing temperature as the CopTer. A non-CopTer primer can be a standard primer (which is any primer that does not have a cooperative anchor structure such as is found in a cooperative primer or tertiary cooperative primer, as defined herein) or a cooperative primer (which has at least one cooperative anchor structure).

A non-nucleotide linker is a chemical moiety that does not allow polymerases to extend synthesis of the complementary strand beyond the linker. Non-limiting examples of non-nucleotide linkers include C3, C6, and C12 spacers, 18-atom non-nucleotide hexa-ethyleneglycol, 9-atom triethylene glycol PEG-150, and abasic tetrahydrofuran (dSpacer).

An “extendable” priming sequence is a segment of nucleotides on the CopTer that anneals to a complementary strand and has a free 3′ end capable of being extended by DNA polymerase. This priming sequence may optionally include base analogs with or without heat-activatable modifications as described by Lebedev et al (2008).

The choice of polymerase depends on the detection method and temperature requirements during amplification, as well as sample preparation conditions immediately prior to amplification. For example, if detection requires separation of a fluorescent label from a quencher by hydrolysis of the reporter molecule (including but not limited to a CopTer also acting as a hydrolysis probe or a conventional Taqman probe), then a DNA polymerase with 5′→3′ exonuclease activity must be used. If hydrolysis of the reporter molecule is not desired for detection, then a DNA polymerase without 5′→3′ exonuclease activity may be used. Both types of DNA polymerases for PCR are well-known in the art and are widely available. For the reverse transcription phase of RT-PCR, the use of RNA-dependent DNA polymerase (also known as reverse transcriptase) that have improved thermostability (such as required if the sample must be kept at high temperature to inhibit RNase activity after sample preparation), or other improved performance characteristics may be required for difficult targets or for increased speed and sensitivity. Non-limiting examples of such reverse transcriptases include engineered heat-tolerant variants of Moloney murine leukemia virus (M-MLV) and avian myelobalstosis virus (AMV) reverse transcriptases as are well-known and available. Naturally thermostable reverse transcriptases such as Codex® HiTemp Reverse Transcriptase (Codexis, Redwood City, CA), and RapiDxFire (Lucigen, Middleton, WI) may also be used.

Double-stranded DNA-binding dyes, are dyes that have very little fluorescence when free, but emits a strong signal when bound to double-stranded DNA. Non-limiting examples of such dyes include SYBR Green I (ThermoFisher Scientific Corporation, Carlsbad, Calif.), EvaGreen (Biotium, Fremont, Calif.), LC Green Plus (BioFire Defense, Salt Lake City, Utah), and Maverick Blue (Co-Diagnostics, Inc., Salt Lake City, Utah). These are discussed in more detail below.

The detection of target nucleic acids can be multiplexed by use of multiple labels, or by differences in melting temperature of amplicons, as are known in the art.

While PCR is the amplification method used in the examples herein, it is understood that any nucleic acid amplification method compatible with the use of primers may use CopTers. Therefore, when the term PCR is used, it should be understood to include such other alternative amplification methods as are known in the art. Methods of amplification, including PCR, are discussed in more detail below.

Specifically, disclosed herein is a tertiary cooperative nucleic acid molecule. This molecule can comprise a first extendable nucleic acid sequence configured to hybridize to a first region of a target nucleic acid; a second nucleic acid sequence located on a 5′ side of the first nucleic acid on the molecule and is non-extendable and configured to hybridize to a second region of the target nucleic acid downstream from a binding site of the first nucleic acid sequence relative to its direction of extension; a first spacer connecting the first and second nucleic acid sequences configured to allow both sequences to hybridize to their targets, and comprising at least one non-nucleotide linker wherein the non-nucleotide linker causes the extension of a complementary strand to terminate; a third nucleic acid sequence located on a 5′ side of the second nucleic acid on the molecule and is non-extendable and configured to hybridize to a third region of the target nucleic acid between where the 3′ end of the first nucleic acid sequence hybridizes and where the 5′ end of the second nucleic acid sequence hybridizes; a second spacer connecting the second and third nucleic acid sequences optionally comprising a non-nucleotide linker. The tertiary cooperative nucleic acid is depicted in FIG. 1A-B.

The first nucleic acid sequence is extendable. This region is represented by “5” in FIGS. 1A and 1B. By “extendable” means that it functions as a 3′ end of a primer, and can be extended by a polymerase. The second and third nucleic acid sequences, also referred to herein as “anchor sequences,” (represented by “3” and “1” in FIGS. 1A and B) are “non-extendable” by a polymerase as they have no free 3′ termini One of skill in the art will appreciate that there are a variety of means to rend these nucleic acid sequences non-extendable. In some embodiments, the non-extendable nucleotides are nucleotide analogs that do not have optimal functional groups for formation of a phosphodiester linkage with another nucleotide. In certain embodiments, the non-extendable nucleotides are chain-terminating nucleotides that allow essentially no primer extension, for example dideoxynucleotides (ddNs), such as ddA, ddC, ddG, ddI, ddT, and ddU. In some embodiments, a polymerase can link other nucleotides to the non-extendable nucleotide, but at slow rate. These are described in more detail in the “definitions” section above.

As noted in FIGS. 1A and 1B, a first spacer (labeled as “4” in FIGS. 1A and 1B) exists between the first (5) and second (3) nucleic acid sequences of the tertiary cooperative primer. This spacer between the first and second nucleic acid sequences can be made of nucleic acids, but must contain a non-nucleotide linker at, or close to the 3′ terminus of the spacer. If the spacer contains nucleotides, it can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or more nucleotides in length, or any number in between. The nucleotides can be naturally occurring or engineered (non-naturally occurring). The spacer may also contain a plurality of non-nucleotide linkers. Types of spacers are discussed elsewhere herein. The spacer can be any length, and can be longer or shorter than the combined length of the first and second nucleic acid sequences, longer or shorter than just the first nucleic acid sequence, or longer or shorter than the second nucleic acid sequence. The spacers can be specifically designed with the target in mind.

There can also be a second spacer (labeled as “2” in FIGS. 1A and 1B) between the second (3) and third (1) nucleic acid sequences. This spacer can have features similar to the first spacer (4), and can be the same length as the first spacer, or can differ in length. In one embodiment, the first spacer (4) is longer than the second spacer (2). In another embodiment, the first (4) and second (2) spacers are the same length. In yet another embodiment, the second (2) spacer is longer than the first (4) spacer. This second spacer may or may not contain a non-nucleotide linker. Spacers are described in more detail below.

Described herein is a tertiary cooperative primer with three regions of nucleic acid sequence which are hybridizable with a target. Also contemplated herein is that there can be more than three regions of the primer which can hybridize with the target nucleic acid. For example, there can be four, five, six, or more regions with a nucleic acid sequence that hybridizes to the target, separated by a third, fourth, or fifth (or more) spacer. In one particular example, the tertiary cooperative primer comprises an additional nucleic acid sequence which comprises at least one non-extendable moiety at its 3′ terminus, wherein said non-extendable moiety renders said additional nucleic acid sequence non-extendable, wherein said additional nucleic acid sequence hybridizes to a noncontiguous segment of the target nucleic acid.

The tertiary cooperative primer must hybridize with the target nucleic acid at the first (5), second (3) and third (1) nucleic acid sequences for amplification to occur. However, this complementarity between the primer and the target need not be 100%. One of skill in the art will recognize that the amount of hybridization can be easily controlled by varying the nucleic acid sequence of the primer, the stringency of the hybridization as controlled by the amplification reaction parameters, etc. The tertiary cooperative primer described herein can have 80, 81, 82, 83, 84, 85, 86, 87, 88, 89, 90, 91, 92, 93, 94, 95, 96, 97, 98, 99, or 100% complementarity where it hybridizes to the target nucleic acid, not including the spacers. In other words, the tertiary cooperative primers can be designed so that 100% complementarity in regions 1, 3, and 5 (referring to FIGS. 1A and 1B) are not needed for amplification to occur. Alternatively, the primer can be designed to maximum stringency, so that 100% complementarity in regions 1, 3, and 5 is required for amplification to occur, or to occur at a rate which is satisfactory to the practitioner.

For hybridization conditions, one of skill in the art will recognize that the specific conditions needed for amplification of a given target can vary. However, in one embodiment, the first nucleic acid sequence (5) cannot hybridize to its complementary target at a temperature that is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C. higher than its own melting temperature, without the second (3) or third (1) nucleic acid sequences also hybridizing to the target. Also contemplated are temperatures in between these amounts, and ranges of these amounts.

In one specific example, the first nucleic acid sequence (5) cannot hybridize to its complementary target at a temperature that is at least 1° C. higher than its own melting temperature, without the anchor sequences, also referred to herein as the second (3) or third (1) nucleic acid sequences, also hybridizing to the target. In yet another specific example, the higher temperature is at least 8° C. higher. In a further example, the higher temperature is between 8° C. and 15° C. higher.

Any of the nucleic acid molecules disclosed herein can have one or more nucleic acid modifications. By “any of the nucleic acid molecules disclosed herein” is meant the nucleic acid sequences designed to hybridize to the target (1, 3, and 5 referring to FIGS. 1A and 1B), or the spacers (2, 4). These modifications can render the molecule non-extendable when desired, as in the case of the second and third nucleic acid sequences and the spacers (1, 2, 3, and 4), or can allow extension to proceed at its 3′ end (as is the case with the first nucleic acid sequence, 5). Such modifications are known to those of skill in the art and are discussed elsewhere herein. They include nucleosides and nucleotide analogs, as defined above in the “definitions” section. The modifications can be at the 5′ terminus, the 3′ terminus, or within the nucleic acid (the “middle” of it). By “terminus” is meant the last nucleotide of that sequence. It can also mean the penultimate sequence, or both.

The target nucleic acid can be a single, continuous molecule. Alternatively, the target nucleic acid can be discontinuous or separated so that the areas of hybridization of the first, second, and third nucleic acid sequence of the tertiary cooperative primer are not contiguous with each other, such as in a highly polymorphic genome wherein long stretches of consensus sequences are hard to find. A non-limiting example of such genome is that of a retrovirus genome. The target nucleic acid can be circular or linear, and can come from any source. The distances between the first and second regions, or the second and third regions on the target can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or more nucleotides in length.

The tertiary cooperative primer can comprise a detectable label. Detectable labels are described in more detail below. The detectable label can be on the first, second, or third (1, 3, or 5) nucleic acid sequence. Alternatively, the detectable label can be on the spacer sequence. The tertiary cooperative primer can comprise more than one label. These labels can be on the same nucleic acid sequence (1, 3, 5) or on the same spacer (2, 4) or can be on more than one, or all, of these sequences. All of these configurations are operable so long as the method allows discrimination between a fee tertiary cooperative primer, and that which has specifically hybridized to the target. In one particular example, the detectable label can comprise a fluorescent resonance energy transfer (FRET) system. For example a double-stranded DNA binding dye in the reaction mixture can act as a fluorescent donor, and a label on the tertiary cooperative primer can act as a fluorescent acceptor; wherein the detection of amplification of the target sequence occurs by illuminating the reaction mixture at an excitation wavelength of the double-stranded DNA binding dye and detecting florescence at a wavelength of emission of the label.

In another embodiment, the detectable label comprises a cleavable moiety. For example, a cleavable moiety contemplated by the present invention is one that produces a detectable signal when hydrolyzed by the 5′ nuclease activity of a thermostable DNA polymerase used for PCR. Again, these are discussed in more detail below.

The tertiary cooperative nucleic acids, such as primers and probes, of this invention are useful in a variety of primer extension/amplification reactions known to those skilled in the art, including, but not limited to the polymerase chain reaction, rolling circle amplification, nucleic acid sequencing and others. The tertiary cooperative primers and probes of this invention can also be used in applications that have post extension/amplification steps, such as hybridization to an array. Because the tertiary cooperative primers/probes in this invention substantially reduce nonspecific hybridization, they are of particular use in multiplexed and highly multiplexed reactions. Specifically, the amplifying step can comprise PCR. In one embodiment, amplifying step can be performed by use of a thermostable DNA polymerase that lacks 5′ to 3′ exonuclease activity. The amplifying step can comprise extension steps that occur at 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C., or any amount in between. In a specific embodiment, the extension steps can occur between 70° C. and 80° C. In a more specific embodiment, the temperature can be between 72° C. and 75° C. The amplifying step can also comprise annealing steps, wherein annealing occurs at 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C. In a specific embodiment, the annealing step takes place between 60° C. and 75° C. In a more specific embodiment, the annealing step occurs between 64° C. and 70° C.

In some embodiments, a single-stranded DNA binding protein is added to the reaction during the step of amplification. to facilitate dissociation of the CopTer's anchor sequences from their complementary amplicon strand.

RT-PCR (reverse transcription PCR) is also contemplated. In one embodiment, reverse transcription precedes amplification. A reverse transcriptase can be included in any kits or methods disclosed herein. The reverse transcription can be performed at 60° C. to 70° C. by a thermostable reverse transcriptase. More details regarding reverse transcriptase are provided below.

In the nucleic acid amplification methods described herein, one of the primers is a tertiary cooperative primer. However, it need not be the case that both primers used are tertiary cooperative primers (although that is also contemplated). The second primer can be selected from a group consisting of a second tertiary cooperative primer (as defined herein), a standard (also referred to herein as a “traditional”, “normal”, or “non-cooperative”) primer, and a cooperative primer, as described in U.S. Pat. No. 10,093,966, herein incorporated by reference in its entirety for its disclosure of cooperative primers.

The use of a tertiary cooperative nucleic acid molecule can decrease the amount of non-specific binding, and/or primer-dimer formation, by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent compared to the amount of primer-dimer, or non-specific binding, present when a normal primer (a non-tertiary cooperative nucleic acid molecule, such as a “standard” or “non-cooperative” primer) is used.

Also disclosed herein is a kit comprising the tertiary cooperative nucleic acid molecules disclosed herein together with instructions for their use. In some embodiments, additional tertiary cooperative nucleic acid molecules, or other primers which are not tertiary cooperative primers, are provided in the kit. In still others, reagents for performing the extension are included, such as polymerase, dNTP's, buffers and the like. In some embodiments, positive and negative controls may be included. In such embodiments, the reagents may all be packaged separately or combined in a single tube or container.

More than one tertiary cooperative nucleic acid molecule can be provided, and they can have the same or different sequences. For example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or more nucleic acid molecules of different sequences can be provided.

### Detailed Embodiments

The following disclosure expands upon the invention described above and provides specific examples, definitions, and embodiments. This section is designed to complement, and expand, the above sections.

### First Nucleic Acid Sequence (Primer) Design

In some embodiments, the isolated melting temperature “Tm” of the primer, also referred to herein as the first nucleic acid sequence, is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more degrees below the reaction temperature used during the annealing phase, of PCR, or the extension phase of reactions with no annealing phase. Therefore, the melting temperature of the primer sequence can be between about 1° C. and 40° C., between about 3° C. and 20° C., between about 5° C. and 15° C. below the reaction temperature used in the PCR reaction. In a preferred embodiment, the isolated Tm is between about 7° C. and 12° C. below the reaction temperature. This provides for less than 50%, and more preferably less than 20% of the template to be hybridized to an isolated primer.

One of skill in the art can design primers with a given melting temperature based on many factors, such as length, GC content, salt, magnesium, potassium and other cosolvents. There are numerous computer programs to derive the melting temperature of an isolated nucleic acid sequence. Optimal designs of the first, second and third sequences that result in an effective tertiary cooperative molecule with the desired effective melting temperature are derived empirically through iterative design as is familiar to those skilled in the art.

To achieve the desired melting temperatures, the first nucleic acid sequence, or the primer (5, referring to FIG. 1A or 1B), can be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 bases in length. For example, the primers can be between about 5 and 26, between about 7 and 22, between about 9 and 17 bases in length depending on GC content.

Any desired number of primers of different nucleotide sequence can be used in the tertiary cooperative primers. The amplification reaction can be performed with, for example, one, two, three, four, five, six, seven, eight, nine, ten, eleven, twelve, thirteen, fourteen, fifteen, sixteen, or seventeen primers. More primers can be used. There is no fundamental upper limit to the number of primers that can be used. However, the use of fewer primers is preferred. When multiple primers are used, the primers should each have a different specific nucleotide sequence.

The amplification reaction can be performed with a single primer and, for example, with no additional primers, with 1 additional primer, with 2 additional primers, with 3 additional primers, with 4 additional primers, with 5 additional primers, with 6 additional primers, with 7 additional primers, with 8 additional primers, with 9 additional primers, with 10 additional primers, with 11 additional primers, with 12 additional primers, with 13 additional primers, with 14 additional primers, with 15 additional primers, with 16 additional primers, with 17 additional primers, with 18 additional primers, with 19 additional primers, with 20 additional primers, with 21 additional primers, with 22 additional primers, with 23 additional primers, with 24 additional primers, with 25 additional primers, with 26 additional primers, with 27 additional primers, with 28 additional primers, with 29 additional primers, with 30 additional primers, with 31 additional primers, with 32 additional primers, with 33 additional primers, with 34 additional primers, with 35 additional primers, with 36 additional primers, with 37 additional primers, with 38 additional primers, with 39 additional primers, with 40 additional primers, with 41 additional primers, with 42 additional primers, with 43 additional primers, with 44 additional primers, with 45 additional primers, with 46 additional primers, with 47 additional primers, with 48 additional primers, with 49 additional primers, with 50 additional primers, with 51 additional primers, with 52 additional primers, with 53 additional primers, with 54 additional primers, with 55 additional primers, with 56 additional primers, with 57 additional primers, with 58 additional primers, with 59 additional primers, with 60 additional primers, with 61 additional primers, with 62 additional primers, with 63 additional primers, with 64 additional primers, with 65 additional primers, with 66 additional primers, with 67 additional primers, with 68 additional primers, with 69 additional primers, with 70 additional primers, with 71 additional primers, with 72 additional primers, with 73 additional primers, with 74 additional primers, with 75 additional primers, with 76 additional primers, with 77 additional primers, with 78 additional primers, with 79 additional primers, with 80 additional primers, with 81 additional primers, with 82 additional primers, with 83 additional primers, with 84 additional primers, with 85 additional primers, with 86 additional primers, with 87 additional primers, with 88 additional primers, with 89 additional primers, with 90 additional primers, with 91 additional primers, with 92 additional primers, with 93 additional primers, with 94 additional primers, with 95 additional primers, with 96 additional primers, with 97 additional primers, with 98 additional primers, with 99 additional primers, with 100 additional primers, with 110 additional primers, with 120 additional primers, with 130 additional primers, with 140 additional primers, with 150 additional primers, with 160 additional primers, with 170 additional primers, with 180 additional primers, with 190 additional primers, with 200 additional primers, with 210 additional primers, with 220 additional primers, with 230 additional primers, with 240 additional primers, with 250 additional primers, with 260 additional primers, with 270 additional primers, with 280 additional primers, with 290 additional primers, with 300 additional primers, with 310 additional primers, with 320 additional primers, with 330 additional primers, with 340 additional primers, with 350 additional primers, with 360 additional primers, with 370 additional primers, with 380 additional primers, with 390 additional primers, with 400 additional primers, with 410 additional primers, with 420 additional primers, with 430 additional primers, with 440 additional primers, with 450 additional primers, with 460 additional primers, with 470 additional primers, with 480 additional primers, with 490 additional primers, with 500 additional primers, with 550 additional primers, with 600 additional primers, with 650 additional primers, with 700 additional primers, with 750 additional primers, with 800 additional primers, with 850 additional primers, with 900 additional primers, with 950 additional primers, with 1,000 additional primers, with 1,100 additional primers, with 1,200 additional primers, with 1,300 additional primers, with 1,400 additional primers, with 1,500 additional primers, with 1,600 additional primers, with 1,700 additional primers, with 1,800 additional primers, with 1,900 additional primers, with 2,000 additional primers, with 2,100 additional primers, with 2,200 additional primers, with 2,300 additional primers, with 2,400 additional primers, with 2,500 additional primers, with 2,600 additional primers, with 2,700 additional primers, with 2,800 additional primers, with 2,900 additional primers, with 3,000 additional primers, with 3,500 additional primers, or with 4,000 additional primers.

The amplification reaction can be performed with a single primer and, for example, with no additional primers, with fewer than 2 additional primers, with fewer than 3 additional primers, with fewer than 4 additional primers, with fewer than 5 additional primers, with fewer than 6 additional primers, with fewer than 7 additional primers, with fewer than 8 additional primers, with fewer than 9 additional primers, with fewer than 10 additional primers, with fewer than 11 additional primers, with fewer than 12 additional primers, with fewer than 13 additional primers, with fewer than 14 additional primers, with fewer than 15 additional primers, with fewer than 16 additional primers, with fewer than 17 additional primers, with fewer than 18 additional primers, with fewer than 19 additional primers, with fewer than 20 additional primers, with fewer than 21 additional primers, with fewer than 22 additional primers, with fewer than 23 additional primers, with fewer than 24 additional primers, with fewer than 25 additional primers, with fewer than 26 additional primers, with fewer than 27 additional primers, with fewer than 28 additional primers, with fewer than 29 additional primers, with fewer than 30 additional primers, with fewer than 31 additional primers, with fewer than 32 additional primers, with fewer than 33 additional primers, with fewer than 34 additional primers, with fewer than 35 additional primers, with fewer than 36 additional primers, with fewer than 37 additional primers, with fewer than 38 additional primers, with fewer than 39 additional primers, with fewer than 40 additional primers, with fewer than 41 additional primers, with fewer than 42 additional primers, with fewer than 43 additional primers, with fewer than 44 additional primers, with fewer than 45 additional primers, with fewer than 46 additional primers, with fewer than 47 additional primers, with fewer than 48 additional primers, with fewer than 49 additional primers, with fewer than 50 additional primers, with fewer than 51 additional primers, with fewer than 52 additional primers, with fewer than 53 additional primers, with fewer than 54 additional primers, with fewer than 55 additional primers, with fewer than 56 additional primers, with fewer than 57 additional primers, with fewer than 58 additional primers, with fewer than 59 additional primers, with fewer than 60 additional primers, with fewer than 61 additional primers, with fewer than 62 additional primers, with fewer than 63 additional primers, with fewer than 64 additional primers, with fewer than 65 additional primers, with fewer than 66 additional primers, with fewer than 67 additional primers, with fewer than 68 additional primers, with fewer than 69 additional primers, with fewer than 70 additional primers, with fewer than 71 additional primers, with fewer than 72 additional primers, with fewer than 73 additional primers, with fewer than 74 additional primers, with fewer than 75 additional primers, with fewer than 76 additional primers, with fewer than 77 additional primers, with fewer than 78 additional primers, with fewer than 79 additional primers, with fewer than 80 additional primers, with fewer than 81 additional primers, with fewer than 82 additional primers, with fewer than 83 additional primers, with fewer than 84 additional primers, with fewer than 85 additional primers, with fewer than 86 additional primers, with fewer than 87 additional primers, with fewer than 88 additional primers, with fewer than 89 additional primers, with fewer than 90 additional primers, with fewer than 91 additional primers, with fewer than 92 additional primers, with fewer than 93 additional primers, with fewer than 94 additional primers, with fewer than 95 additional primers, with fewer than 96 additional primers, with fewer than 97 additional primers, with fewer than 98 additional primers, with fewer than 99 additional primers, with fewer than 100 additional primers, with fewer than 110 additional primers, with fewer than 120 additional primers, with fewer than 130 additional primers, with fewer than 140 additional primers, with fewer than 150 additional primers, with fewer than 160 additional primers, with fewer than 170 additional primers, with fewer than 180 additional primers, with fewer than 190 additional primers, with fewer than 200 additional primers, with fewer than 210 additional primers, with fewer than 220 additional primers, with fewer than 230 additional primers, with fewer than 240 additional primers, with fewer than 250 additional primers, with fewer than 260 additional primers, with fewer than 270 additional primers, with fewer than 280 additional primers, with fewer than 290 additional primers, with fewer than 300 additional primers, with fewer than 310 additional primers, with fewer than 320 additional primers, with fewer than 330 additional primers, with fewer than 340 additional primers, with fewer than 350 additional primers, with fewer than 360 additional primers, with fewer than 370 additional primers, with fewer than 380 additional primers, with fewer than 390 additional primers, with fewer than 400 additional primers, with fewer than 410 additional primers, with fewer than 420 additional primers, with fewer than 430 additional primers, with fewer than 440 additional primers, with fewer than 450 additional primers, with fewer than 460 additional primers, with fewer than 470 additional primers, with fewer than 480 additional primers, with fewer than 490 additional primers, with fewer than 500 additional primers, with fewer than 550 additional primers, with fewer than 600 additional primers, with fewer than 650 additional primers, with fewer than 700 additional primers, with fewer than 750 additional primers, with fewer than 800 additional primers, with fewer than 850 additional primers, with fewer than 900 additional primers, with fewer than 950 additional primers, with fewer than 1,000 additional primers, with fewer than 1,100 additional primers, with fewer than 1,200 additional primers, with fewer than 1,300 additional primers, with fewer than fewer than 1,400 additional primers, with fewer than 1,500 additional primers, with fewer than 1,600 additional primers, with fewer than 1,700 additional primers, with fewer than 1,800 additional primers, with fewer than 1,900 additional primers, with fewer than 2,000 additional primers, with fewer than 2,100 additional primers, with fewer than 2,200 additional primers, with fewer than 2,300 additional primers, with fewer than 2,400 additional primers, with fewer than 2,500 additional primers, with fewer than 2,600 additional primers, with fewer than 2,700 additional primers, with fewer than 2,800 additional primers, with fewer than 2,900 additional primers, with fewer than 3,000 additional primers, with fewer than 3,500 additional primers, or with fewer than 4,000 additional primers.

The amplification reaction can be performed, for example, with fewer than 2 primers, with fewer than 3 primers, with fewer than 4 primers, with fewer than 5 primers, with fewer than 6 primers, with fewer than 7 primers, with fewer than 8 primers, with fewer than 9 primers, with fewer than 10 primers, with fewer than 11 primers, with fewer than 12 primers, with fewer than 13 primers, with fewer than 14 primers, with fewer than 15 primers, with fewer than 16 primers, with fewer than 17 primers, with fewer than 18 primers, with fewer than 19 primers, with fewer than 20 primers, with fewer than 21 primers, with fewer than 22 primers, with fewer than 23 primers, with fewer than 24 primers, with fewer than 25 primers, with fewer than 26 primers, with fewer than 27 primers, with fewer than 28 primers, with fewer than 29 primers, with fewer than 30 primers, with fewer than 31 primers, with fewer than 32 primers, with fewer than 33 primers, with fewer than 34 primers, with fewer than 35 primers, with fewer than 36 primers, with fewer than 37 primers, with fewer than 38 primers, with fewer than 39 primers, with fewer than 40 primers, with fewer than 41 primers, with fewer than 42 primers, with fewer than 43 primers, with fewer than 44 primers, with fewer than 45 primers, with fewer than 46 primers, with fewer than 47 primers, with fewer than 48 primers, with fewer than 49 primers, with fewer than 50 primers, with fewer than 51 primers, with fewer than 52 primers, with fewer than 53 primers, with fewer than 54 primers, with fewer than 55 primers, with fewer than 56 primers, with fewer than 57 primers, with fewer than 58 primers, with fewer than 59 primers, with fewer than 60 primers, with fewer than 61 primers, with fewer than 62 primers, with fewer than 63 primers, with fewer than 64 primers, with fewer than 65 primers, with fewer than 66 primers, with fewer than 67 primers, with fewer than 68 primers, with fewer than 69 primers, with fewer than 70 primers, with fewer than 71 primers, with fewer than 72 primers, with fewer than 73 primers, with fewer than 74 primers, with fewer than 75 primers, with fewer than 76 primers, with fewer than 77 primers, with fewer than 78 primers, with fewer than 79 primers, with fewer than 80 primers, with fewer than 81 primers, with fewer than 82 primers, with fewer than 83 primers, with fewer than 84 primers, with fewer than 85 primers, with fewer than 86 primers, with fewer than 87 primers, with fewer than 88 primers, with fewer than 89 primers, with fewer than 90 primers, with fewer than 91 primers, with fewer than 92 primers, with fewer than 93 primers, with fewer than 94 primers, with fewer than 95 primers, with fewer than 96 primers, with fewer than 97 primers, with fewer than 98 primers, with fewer than 99 primers, with fewer than 100 primers, with fewer than 110 primers, with fewer than 120 primers, with fewer than 130 primers, with fewer than 140 primers, with fewer than 150 primers, with fewer than 160 primers, with fewer than 170 primers, with fewer than 180 primers, with fewer than 190 primers, with fewer than 200 primers, with fewer than 210 primers, with fewer than 220 primers, with fewer than 230 primers, with fewer than 240 primers, with fewer than 250 primers, with fewer than 260 primers, with fewer than 270 primers, with fewer than 280 primers, with fewer than 290 primers, with fewer than 300 primers, with fewer than 310 primers, with fewer than 320 primers, with fewer than 330 primers, with fewer than 340 primers, with fewer than 350 primers, with fewer than 360 primers, with fewer than 370 primers, with fewer than 380 primers, with fewer than 390 primers, with fewer than 400 primers, with fewer than 410 primers, with fewer than 420 primers, with fewer than 430 primers, with fewer than 440 primers, with fewer than 450 primers, with fewer than 460 primers, with fewer than 470 primers, with fewer than 480 primers, with fewer than 490 primers, with fewer than 500 primers, with fewer than 550 primers, with fewer than 600 primers, with fewer than 650 primers, with fewer than 700 primers, with fewer than 750 primers, with fewer than 800 primers, with fewer than 850 primers, with fewer than 900 primers, with fewer than 950 primers, with fewer than 1,000 primers, with fewer than 1,100 primers, with fewer than 1,200 primers, with fewer than 1,300 primers, with fewer than fewer than 1,400 primers, with fewer than 1,500 primers, with fewer than 1,600 primers, with fewer than 1,700 primers, with fewer than 1,800 primers, with fewer than 1,900 primers, with fewer than 2,000 primers, with fewer than 2,100 primers, with fewer than 2,200 primers, with fewer than 2,300 primers, with fewer than 2,400 primers, with fewer than 2,500 primers, with fewer than 2,600 primers, with fewer than 2,700 primers, with fewer than 2,800 primers, with fewer than 2,900 primers, with fewer than 3,000 primers, with fewer than 3,500 primers, or with fewer than 4,000 primers.

As discussed above, the disclosed primers can have one or more modified nucleotides. Such primers are referred to herein as modified primers. Chimeric primers can also be used. Chimeric primers are primers having at least two types of nucleotides, such as both deoxyribonucleotides and ribonucleotides, ribonucleotides and modified nucleotides, two or more types of modified nucleotides, deoxyribonucleotides and two or more different types of modified nucleotides, ribonucleotides and two or more different types of modified nucleotides, or deoxyribonucleotides, ribonucleotides and two or more different types of modified nucleotides. One form of chimeric primer is peptide nucleic acid/nucleic acid primers. For example, 5′-PNA-DNA-3′ or 5′-PNA-RNA-3′ primers may be used for more efficient strand invasion and polymerization invasion. Other forms of chimeric primers are, for example, 5′-(2′-O-Methyl) RNA-RNA-3′ or 5′-(2′-O-Methyl) RNA-DNA-3′.

Many modified nucleotides (nucleotide analogs) are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and 0-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. A primer having one or more universal bases is not considered to be a primer having a specific sequence.

Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O—, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n-ONH₂, and —O(CHO)nON[(CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to complementary nucleic acids in a Watson-Crick or Hoogsteen manner, but which are linked together through a moiety other than a phosphate moiety. Nucleotide substitutes are able to conform to a double helix type structure when interacting with the appropriate nucleic acid molecules.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Primers can be comprised of nucleotides and can be made up of different types of nucleotides or the same type of nucleotides. For example, one or more of the nucleotides in a primer can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. The nucleotides can be comprised of bases (that is, the base portion of the nucleotide) and can (and normally will) comprise different types of bases.

### Anchor Sequence Design (Second and Third Nucleic Acid Sequence)

The anchor sequences, also referred to herein as the “second nucleic acid sequence” and “third nucleic acid sequence” (1 and 3 in FIGS. 1A and 1B) are complementary to the template such that it hybridizes to the target nucleic acid molecule downstream from the 3′ end of the primer. In some embodiments, resistance to mutations in the target nucleic acid is desired and the anchor sequence is designed with a melting temperature greater than the annealing temperature. In these embodiments, the anchor sequence is designed with an isolated Tm of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more degrees above the annealing or extension (“reaction”) temperature. For example, the anchor, or second and third, sequences are between about 0° C. and 40° C., between about 5° C. and 30° C., between about 7° C. and 25° C. above the reaction temperature. In some embodiments, the predicted melting temperature of the anchor sequence is also made for expected mutants. In these embodiments, the isolated Tm of the anchor sequences to the expected mutants is between about 1, 2, 3, 4, 5, 6, 7, 8, 9, 10, 11, 12, 13, 14, 15 16, 17, 18, 19, 20, or more degrees C. below the reaction temperature, or 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 or more degrees C. above the reaction temperature. For example, it can be 10° C. below the reaction temperature and 30° C. above the reaction temperature, between about 3° C. below the reaction temperature and about 10° C. above the reaction temperature.

To achieve these melting temperatures, the anchor sequence length can be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 or more bases in length. For example, it can be between about 20 and about 50, between about 22 and about 40, between about 23 and about 37 bases. It is noted that the second and third anchor sequences can differ in length from each other.

In some embodiments, an even higher resistance to mutations in the target sequence is desired. In these embodiments, in addition to an anchor sequence with an isolated Tm of between about 0° C. and 40° C. above the reaction temperature, the tertiary cooperative primer is designed with an isolated Tm of between about 7° C. below and about 20° C. above, between about 5° C. below and about 10° C. above, between about 3° C. below and about 3° C. above the reaction temperature. The tertiary cooperative interaction between the primer and the anchor sequence will result in an even greater effective Tm for the tertiary cooperative primer, rendering it almost impervious to mutations in the sequence. By comparison, a normal (or standard, “non-cooperative”) primer might have to be an additional 5 to 30 bases in length to have an equivalent resistance to mutations in the target sequence, and consequently, would be much more susceptible to primer-dimer formation.

In other embodiments, a higher resistance to primer-dimers is preferred and the melting temperature of the isolated anchor, or second or third, nucleic acid sequence is designed to be less than the reaction temperature. For example, the anchor, or second or third, nucleic acid sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more degrees below the reaction temperature, or annealing phase, of PCR. In preferred embodiments, the Tm of the isolated anchor, or second or third nucleic acid, sequence is between about 0° C. and 12° C., between about 1° C. and 8° C., between about 2° C. and 5° C. below the reaction temperature. To achieve these low melting temperatures, the anchor sequence length can be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more bases in length. For example, the anchor, or second or third nucleic acid sequence, can be between about 5 and 30, between about 8 and 25, and between about 10 and 22 bases.

In some embodiments, the anchor sequences bind and releases the target sequence rapidly such that the polymerase can extend underneath the anchor sequences, leaving the anchor sequences intact. In some embodiments, this is enhanced using a tertiary cooperative primer with the spacer attached to the 5′ end of one or more of the anchor sequences. In a preferred embodiment, the polymerase is capable of cleaving one or more of the anchor sequences during extension. In a preferred embodiment, this is enhanced using a tertiary cooperative primer with the spacer attached to the 3′ end of one or more of the anchor sequences.

### Spacer Sequences

The spacer sequences are designated as 2 and 4 in FIGS. 1A and 1B. The following description applies to both spacers, although they can differ from one another. The number of bases between the 3′ end of the first nucleic acid, or primer, sequence and the 5′ end of the second or third nucleic acid, or anchor sequence hybridization locations in the template is important. In some embodiments, the number of bases between the primer and the anchor sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. For example, they can be between about 0 and 30, between about 0 and 20, between about 0 and 10 bases.

In some embodiments, the spacer attaches the 5′ end of the primer to the 3′ end of one or both anchor sequences. In a preferred embodiment where the spacer attaches to the 3′ end of the anchor sequence, the spacer comprises an 18-atom hexa-ethyleneglycol. In another embodiment, the primer is inverted such that the 5′ end of the primer is attached to the 5′ end of both anchor sequences. In this embodiment, the spacer is longer than the primer. In a preferred embodiment where the spacer attaches to the 5′ end of the anchor sequence, the spacer comprises 3 hexaethylene glycols. In yet another embodiment, the 3′ end of the capture sequence is linked to the middle of the primer. In this instance, the spacer may be shorter than the length of the primer.

A variety of spacer types and compositions are known to those skilled in the art which can be used as the spacers disclosed herein. Examples include, but are not limited to, polyethylene glycol and carbon linkers. Linkers can be attached through a variety of methods, including but not limited to, covalent bonds, ionic bonds, hydrogen bonding, polar association, magnetic association, and van der wals association. A preferred method is covalent bonding through standard DNA synthesis methods.

The length of polyethylene glycol linkers is about 0.34 nm per monomer. In some embodiments, the length of the polyethylene glycol linker is between about 1 and 90, between about 2 and 50, between about 3 and 30 monomers (between about 1 and 10 nm fully extended).

### Probes

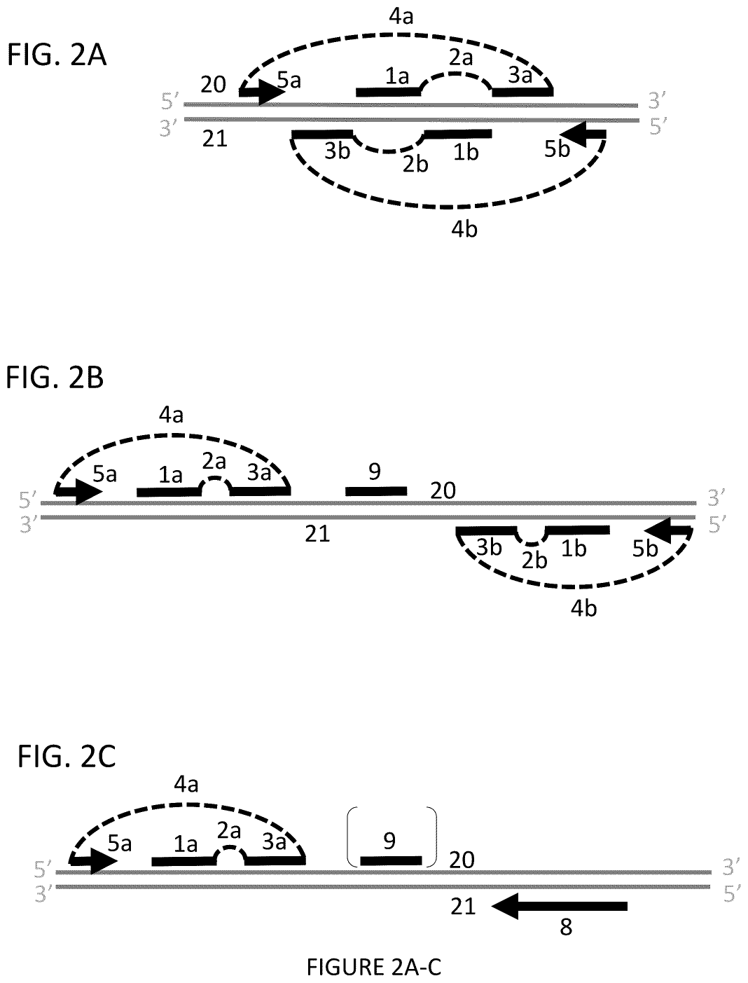

In some embodiments, a probe is used which is a separate nucleic acid molecule. This can be seen in FIGS. 2B and 2C, which depicts “9” as a separate probe (not part of the tertiary cooperative primer). One of skill in the art will understand how to use probes to detect the presence of nucleic acids.

In another embodiment, the anchor sequence (the second and/or third nucleic acid sequence of the tertiary cooperative primer, 3 and/or 1) also serve as a probe. In some embodiments, this is done through the addition of one or more labels to the anchor sequence or sequences. In a preferred embodiment, the labels include a FRET pair.

Various nucleic acid probe constructs are known to those skilled in the art. These include, but are not limited to, dual labeled probes, hairpin probes, and single label probes.

In some embodiments, a low background signal is desired for high signal to noise ratios. In some embodiments, a hairpin probe is used to provide increased contact quenching to assist in providing high signal to noise.

In other embodiments, a shorter probe is desired to minimize primer-probe dimers. In some embodiments requiring a shorter probe, a dual labeled probe is used. In embodiments that require an even greater emphasis on the reduction of spurious extension products, the melting temperature of the isolated probe target complex is less than the reaction temperature.

A variety of methods for detecting signal from labeled probes are known to those skilled in the art. In some embodiments a polymerase is used that cleaves the probe, releasing a label that changes the signal. In other embodiments, a polymerase is used that does not cleave the probe. Rather the signal is modified by the hybridization of the probe to the template.

Using a Primer with a Built in Detection Mechanism

In some embodiments, the primer has a built in detection mechanism. In some embodiments the detection mechanism includes one or more detectable labels. In a preferred embodiment, the detection mechanism includes a FRET pair. Examples of primers with built in detection mechanisms include, but are not limited to, Amplifluor primers, Rapid Detex primers, and others known to those skilled in the art.

Tertiary cooperative nucleic acid primers with built in detection mechanisms can be more useful to assay designers than non-cooperative nucleic acids (normal, or standard, “non-cooperative” primers) with built in detection mechanisms. Without being limited by theory, this is because cooperative nucleic acids are less prone to generate signal from nonspecific products, such as primer-dimers.

In some embodiments, a double-stranded nucleic acid binding dye, such as SYBR Green, is used to monitor the progress of the amplification reaction.

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers. The use of several types of fluorescent change probes and primers are reviewed in Schweitzer and Kingsmore, Curr. Opin. Biotech. 12:21-27 (2001). Hall et al., Proc. Natl. Acad. Sci. USA 97:8272-8277 (2000), describe the use of fluorescent change probes with Invader assays.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TaqMan probes (Holland et al., Proc. Natl. Acad. Sci. USA 88:7276-7280 (1991)) are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends a the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers (Nazerenko et al., Nucleic Acids Res. 25:2516-2521 (1997)) and scorpion primers (Thelwell et al., Nucleic Acids Res. 28(19):3752-3761 (2000)).

Cleavage activated primers are similar to cleavage activated probes except that they are primers that are incorporated into replicated strands and are then subsequently cleaved. Little et al., Clin. Chem. 45:777-784 (1999), describe the use of cleavage activated primers.

Multiplexing with ARMS

In some embodiments, detection of multiple polymorphisms, insertions, deletions or other mutations is desired. In some embodiments, the primer is designed such that the base on the 3′ end is over the mutation. In some embodiments, additional intentional polymorphisms are designed into the primer. In one embodiment, the presence of a probe attached to the primer allows for allele specific real-time detection of multiple polymorphisms in the same location.

Mutation Differentiation with the Probe

In some embodiments the differentiation of polymorphisms is accomplished using the tertiary cooperative primer. In some embodiments the anchor sequence or sequences has additional mutations intentionally added to improve differentiation. In some embodiments, the anchor sequences will not bind when a polymorphism is present, preventing efficient amplification round after around. In some embodiments where the anchor sequence has a detectable label, even if some amplification does occur, the anchor sequence does not bind sufficiently to generate a detectable signal.

### RNA and Other Reactions

In some embodiments, a variety of polymerases and enzymes capable of adding one or more bases to a nucleic acid template as are known to those skilled in the art are used. In some embodiments, reverse transcription is desired. In some embodiments, the probe has a sufficiently low melting temperature that the polymerase can extend by displacing it. In other embodiments, an increase in the temperature after a time for initial polymerization removes the anchor sequences from the template, allowing the polymerase to extend. In other embodiments, additional primer sequences are used that do not have a anchor sequence, allowing the polymerase to make copies in an uninhibited fashion at lower reaction temperatures.

### Target Nucleic Acid Molecules

Nucleic acid molecules, which are the object of amplification, can be any nucleic acid from any source. In general, the disclosed method is performed using a nucleic acid sample that contains (or is suspected of containing) nucleic acid molecules to be amplified.

A nucleic acid sample can be any nucleic acid sample of interest. The source, identity, and preparation of many such nucleic acid samples are known. It is preferred that nucleic acid samples known or identified for use in amplification or detection methods be used for the method described herein. The nucleic acid sample can be, for example, a nucleic acid sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, and archeological samples such as bone or mummified tissue. Types of useful nucleic acid samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, a crude cell lysate samples, forensic samples, archeological samples, infection samples, nosocomial infection samples, production samples, drug preparation samples, biological molecule production samples, protein preparation samples, lipid preparation samples, and/or carbohydrate preparation samples.

For whole genome amplification, preferred nucleic acid samples are nucleic acid samples from a single cell. The nucleic acid samples for use in the disclosed method are preferably nucleic acid molecules and samples that are complex and non-repetitive. Where the nucleic acid sample is a genomic nucleic acid sample, the genome can be the genome from any organism of interest. For example, the genome can be a viral genome, a bacterial genome, a eubacterial genome, an archae bacterial genome, a fungal genome, a microbial genome, a eukaryotic genome, a plant genome, an animal genome, a vertebrate genome, an invertebrate genome, an insect genome, a mammalian genome, or a human genome. The target genome is preferably pure or substantially pure, but this is not required. For example, an genomic sample from an animal source may include nucleic acid from contaminating or infecting organisms.

The nucleic acid sample can be, or can be derived from, for example, one or more whole genomes from the same or different organisms, tissues, cells or a combination; one or more partial genomes from the same or different organisms, tissues, cells or a combination; one or more whole chromosomes from the same or different organisms, tissues, cells or a combination; one or more partial chromosomes from the same or different organisms, tissues, cells or a combination; one or more chromosome fragments from the same or different organisms, tissues, cells or a combination; one or more artificial chromosomes; one or more yeast artificial chromosomes; one or more bacterial artificial chromosomes; one or more cosmids; or any combination of these.

### Oligonucleotide Synthesis

Primers, detection probes, address probes, and any other oligonucleotides can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method. Solid phase chemical synthesis of DNA fragments is routinely performed using protected nucleoside cyanoethyl phosphoramidites (S. L. Beaucage et al. (1981) Tetrahedron Lett. 22:1859). In this approach, the 3′-hydroxyl group of an initial 5′-protected nucleoside is first covalently attached to the polymer support (R. C. Pless et al. (1975) Nucleic Acids Res. 2:773 (1975)). Synthesis of the oligonucleotide then proceeds by deprotection of the 5′-hydroxyl group of the attached nucleoside, followed by coupling of an incoming nucleoside-3′-phosphoramidite to the deprotected hydroxyl group (M. D. Matteucci et a. (1981) J. Am. Chem. Soc. 103:3185). The resulting phosphite triester is finally oxidized to a phosphorotriester to complete the internucleotide bond (R. L. Letsinger et al. (1976) J. Am. Chem. Soc. 9:3655). Alternatively, the synthesis of phosphorothioate linkages can be carried out by sulfurization of the phosphite triester. Several chemicals can be used to perform this reaction, among them 3H-1,2-benzodithiole-3-one, 1,1-dioxide (R. P. Iyer, W. Egan, J. B. Regan, and S. L. Beaucage, J. Am. Chem. Soc., 1990, 112, 1253-1254). The steps of deprotection, coupling and oxidation are repeated until an oligonucleotide of the desired length and sequence is obtained. Other methods exist to generate oligonucleotides such as the H-phosphonate method (Hall et al, (1957) J. Chem. Soc., 3291-3296) or the phosphotriester method as described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994). Other forms of oligonucleotide synthesis are described in U.S. Pat. Nos. 6,294,664 and 6,291,669.

So long as their relevant function is maintained, primers, detection probes, address probes, and any other oligonucleotides can be made up of or include modified nucleotides (nucleotide analogs). Many modified nucleotides are known and can be used in oligonucleotides, and are disclosed elsewhere herein.

### Kits

The materials described above as well as other materials can be combined together in any suitable configuration useful for performing, or aiding in the performance of, the disclosed method. It is useful if the kit components in a given kit are designed and adapted for use together in the disclosed method. For example, disclosed are kits for amplification of nucleic acid samples, the kit comprising tertiary cooperative nucleic acids and a DNA polymerase. The kits also can contain nucleotides, buffers, detection probes, fluorescent change probes, lysis solutions, stabilization solutions, denaturation solutions, or a combination.

Specifically, contemplated herein is a kit for amplification, wherein the kit comprises:
a) a tertiary cooperative primer according to the primers disclosed herein; and
b) a thermostable polymerase.
   The kit further can comprise additional primers, such as one or more additional tertiary cooperative primer. The additional primer or primers can also be cooperative primers or non-cooperative primers. The kit can further comprise reagents useful for the amplification of nucleic acid. These reagents can be in an amplification buffer. The kit can further comprise a double-stranded DNA binding dye. In one embodiment, the tertiary cooperative primer is labeled with one or more fluorescent labels and optionally with one or more quenchers. One or more thermostable polymerases can be selected from a group consisting of thermostable DNA polymerase and thermostable reverse transcriptase. The kit can comprise a reaction mixture. Also disclosed are reaction mixtures themselves.

### Uses

The disclosed method and compositions are applicable to numerous areas including, but not limited to, analysis of nucleic acids present in cells and viruses, for medical, research, education, home, and environmental testing and screening.

### Amplification

It will be appreciated that while PCR is the amplification method used in the examples herein, it is understood that any amplification method that uses a primer may be suitable, whether the signal or target is amplified. In fact, any proximity-based amplification approaches known to those of skill in that art may be used, including assays for the signal amplification to detect antigens. Some suitable procedures may include polymerase chain reaction (PCR); strand displacement amplification (SDA); nucleic acid sequence-based amplification (NASBA); cascade rolling circle amplification (CRCA), loop-mediated isothermal amplification of DNA (LAMP); isothermal and chimeric primer-initiated amplification of nucleic acids (ICAN); target based-helicase dependent amplification (HDA); transcription-mediated amplification (TMA), CRISPR-Cas9-triggered strand displacement amplification, immuno-PCR, recombinase polymerase assay (RPA) and the like. Amplification methods may further include analyses such as melting curve analysis. Therefore, when the term amplification is used, it should be understood to include other alternative amplification methods and analysis of amplification products. It is understood that protocols may need to be adjusted accordingly.

The various sequence representations described above and elsewhere herein can be, for example, for 1 target sequence, 2 target sequences, 3 target sequences, 4 target sequences, 5 target sequences, 6 target sequences, 7 target sequences, 8 target sequences, 9 target sequences, 10 target sequences, 11 target sequences, 12 target sequences, 13 target sequences, 14 target sequences, 15 target sequences, 16 target sequences, 17 target sequences, 18 target sequences, 19 target sequences, 20 target sequences, 25 target sequences, 30 target sequences, 40 target sequences, 50 target sequences, 75 target sequences, or 100 target sequences. The sequence representation can be, for example, for at least 1 target sequence, at least 2 target sequences, at least 3 target sequences, at least 4 target sequences, at least 5 target sequences, at least 6 target sequences, at least 7 target sequences, at least 8 target sequences, at least 9 target sequences, at least 10 target sequences, at least 11 target sequences, at least 12 target sequences, at least 13 target sequences, at least 14 target sequences, at least 15 target sequences, at least 16 target sequences, at least 17 target sequences, at least 18 target sequences, at least 19 target sequences, at least 20 target sequences, at least 25 target sequences, at least 30 target sequences, at least 40 target sequences, at least 50 target sequences, at least 75 target sequences, or at least 100 target sequences.

The sequence representation can be, for example, for 1 target sequence, 2 different target sequences, 3 different target sequences, 4 different target sequences, 5 different target sequences, 6 different target sequences, 7 different target sequences, 8 different target sequences, 9 different target sequences, 10 different target sequences, 11 different target sequences, 12 different target sequences, 13 different target sequences, 14 different target sequences, 15 different target sequences, 16 different target sequences, 17 different target sequences, 18 different target sequences, 19 different target sequences, 20 different target sequences, 25 different target sequences, 30 different target sequences, 40 different target sequences, 50 different target sequences, 75 different target sequences, or 100 different target sequences. The sequence representation can be, for example, for at least 1 target sequence, at least 2 different target sequences, at least 3 different target sequences, at least 4 different target sequences, at least 5 different target sequences, at least 6 different target sequences, at least 7 different target sequences, at least 8 different target sequences, at least 9 different target sequences, at least 10 different target sequences, at least 11 different target sequences, at least 12 different target sequences, at least 13 different target sequences, at least 14 different target sequences, at least 15 different target sequences, at least 16 different target sequences, at least 17 different target sequences, at least 18 different target sequences, at least 19 different target sequences, at least 20 different target sequences, at least 25 different target sequences, at least 30 different target sequences, at least 40 different target sequences, at least 50 different target sequences, at least 75 different target sequences, or at least 100 different target sequences.

### Detection

Products of amplification can be detected using any nucleic acid detection technique. For real-time detection, the amplification products and the progress of amplification are detected during amplification. Real-time detection is usefully accomplished using one or more or a combination of fluorescent change probes and fluorescent change primers. Other detection techniques can be used, either alone or in combination with real-timer detection and/or detection involving fluorescent change probes and primers. Many techniques are known for detecting nucleic acids. The nucleotide sequence of the amplified sequences also can be determined using any suitable technique. It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### EXAMPLES

### Example 1

Amplification of P. gingivalis with a CopTer Paired with a Standard Primer and Detected with a Double-Stranded DNA-Binding Dye.

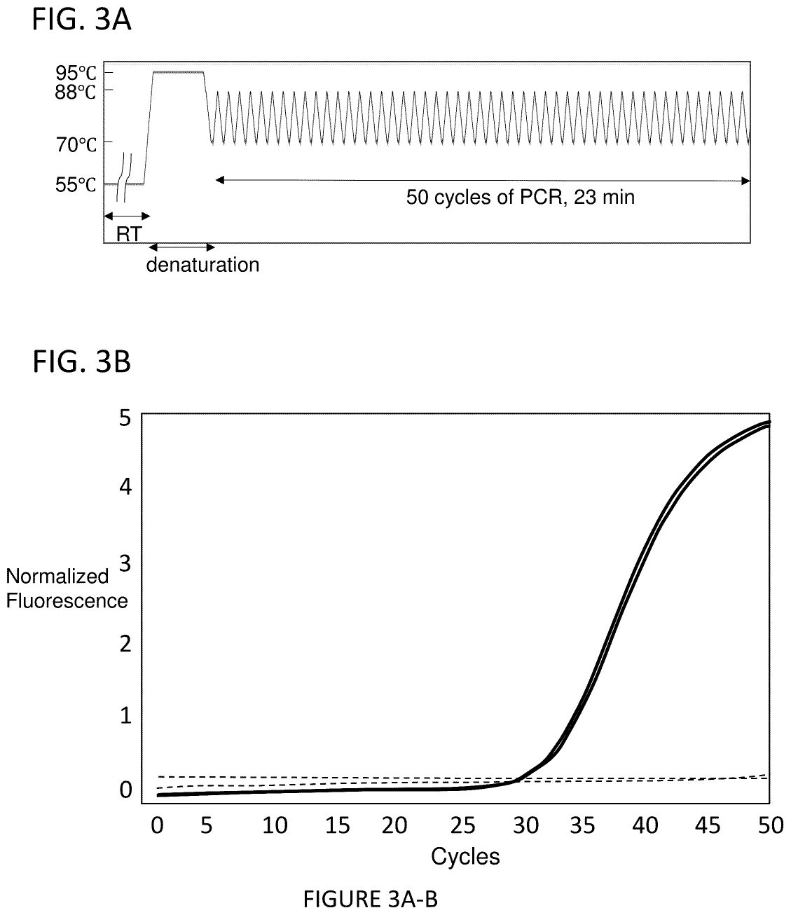

A 111 bp fragment (Tm=85° C.) of the hmuY gene of gram-negative bacteria Porphyromonas gingivalis was amplified with forward primer 5′ GGCAGGACCGTGAGAGAATTCC3′ (SEQ ID NO: 1) (Tm=69° C.) and reverse CopTer 5′ AGTGATTACCGGCAAGAAGAACGCA (SEQ ID NO: 2) /iSp18/ATTTGCTTCAGGTGGTTGGCT (SEQ ID NO: 3) /iSp18/TGGAATATGAAGAACAGGGCTTC3′ (SEQ ID NO: 4) in the presence of synthetic RNA template 5′ CGGCAGGACCGUGAGAGAAUUCCAGCCAACCACCUGAAGCAAAUCUAGGUA AGUCUAUGUGCGUUCUUCUUGCCGGUAAUCACUUCGCUGAAGCCCUGUUCUU CAUAUUCCAU3′ (SEQ ID NO: 5). An 18-atom non-nucleotide hexa-ethyleneglycol spacer is represented by “iSP18”. Primer and CopTer binding sites or their complementary sequences are underlined. Similarly, a 102 bp fragment (Tm=83.5° C.) of the hmuY gene was amplified with forward primer 5′ CGGCAGGACCGTGAGAGAATTC3′ (SEQ ID NO: 6) (Tm=69° C.) and the aforementioned CopTer in the presence of synthetic DNA template 5′ GACCTGATGGTCATCAGATGGAATATGAAGAACAGGGCTTCAGCGAAGTGATT ACCGGCAAGAAGAACGCACAGGGATTTGCTTCAGGTGGTTGGCTGGAATTCTCT CACGGTCCTGCCGGTCCC3′ (SEQ ID NO: 7). CopTers, primers, and templates were all obtained from Integrated DNA Technologies (Coralville, Iowa). The reaction mixture included 20,000 copies of RNA template, 0.25 μM forward primer, 0.25 μM reverse CopTer, 1× EvaGreen (Biotium, Fremont, Calif.), 1× Preformulated PCR Master Mix E (Promega, Madison, Wisconsin) supplemented with 5 mM MgCl₂ (Sigma-Aldrich, St. Louis, Missouri), 0.156 U/μL GoTaq MDx Hot Start Polymerase (Promega), and 3.2 U/μL GoScript reverse transcriptase (Promega) when amplifying from RNA template only. Samples were 10 μL each, placed in MIC 4-tube strips (MBS Biosystems, Houston, Texas), and were temperature cycled on a Magnetic Induction Cycler (Bio Molecular Systems, Upper Coomera, Australia) with or without a reverse transcription step (55° C. for 10 mM and 95° C. for 120 s if RNA template was used), followed by denaturation at 95° C. for 20 s, and then 50 cycles of temperature cycling between 88° C. for 1 s and 70° C. for 2 s. Fluorescence was collected at 70° C. every cycle in the Green channel (excitation 465 nm, detection 510 nm). FIG. 3A shows the temperature cycling profile of 50 cycles which was completed in 23 mM, and FIG. 3B shows robust amplification curves generated from duplicate samples of RNA template. The no-template controls (water) showed no amplification. Fast temperature cycling was possible due to the narrow temperature difference between denaturation at 88° C. and annealing at 70° C. Melting curve analysis performed on the amplified product (data not shown) confirmed a single peak at the expected melting temperature of the amplicon, confirming high specificity of the amplification reaction. Specific amplification products were also obtained with the DNA template with 50 cycles of PCR completed in less than 25 minutes (not shown).

### Example 2

Amplification of Pepper Mild Mottle Virus (PMMV) with a CopTer Primer Pair and Detected with a Double-Stranded DNA Binding Dye.

RNA from Pepper Mild Mottle Virus (PMMV) was isolated from Tabasco sauce (McIlhenny Company, New Orleans, LA) using Quick-RNA kit (Zymo Research Corp, Irvine, CA). This RNA was used as template to compare the performance of CopTer primer pairs against standard primer pairs and cooperative primer pairs in real time reverse transcription PCR. Each of the primer sequences are shown in Table 1.

TABLE 1

| Type of | | |
|---|---|---|
| Primer | ID | Sequence (5′ to 3′) |
| CopTer | S01 | TTCAAATGAGAGTGGT (SEQ ID NO: 8) |
| | | /iSp18/TGTCGCACTTGCATTGCA (SEQ ID NO: 9) |
| | | /iSp18/GGCGTAGATCC (SEQ ID NO: 10) |
| CopTer | S02 | TTGCTTCGGTAGG (SEQ ID NO: 11) |
| | | /iSp18/GGCTACCATTACCTTTGCTGC (SEQ ID NO: 12) |
| | | /iSp18/GCAATTGTCGG (SEQ ID NO: 13) |
| Cooperative | S03 | TGGTGGCAGCA (SEQ ID NO: 14) |
| | | /iSp18/AAAAAA (SEQ ID NO: 15) |
| | | /iSp18/GGCGTAGATCCA (SEQ ID NO: 16) |
| Cooperative | S04 | ATGCAAGTGCGAC (SEQ ID NO: 17) |
| | | /iSp18/AAAAAA (SEQ ID NO: 15) |
| | | /iSp18/AATTGTCGGTTGC (SEQ ID NO: 18) |
| Standard | S05 | GCAGCAAAGGTAATGGTAGC (SEQ ID NO: 19) |
| Standard | S06 | TGCAAGTGCGACATTTGCTTCGGT (SEQ ID NO: 20) |

The estimated melting temperatures of each annealing sequences are shown in Table 2. Anchor 1 is the 5′ most non-extending annealing sequence on the primer molecule (if present), and Anchor 2 is the non-extending annealing sequence located between the priming sequence and Anchor 1 (if present).

TABLE 2

| | | Estimated Melting Temperatures of | | |
|---|---|---|---|---|
| Type of | | Priming | Anchor 1 | Anchor 2 |
| Primer | ID | sequence | sequence | sequence |
| CopTer | S01 | 45.0° C. | 52.9° C. | 65.1° C. |
| CopTer | S02 | 43.4° C. | 52.1° C. | 65.1° C. |
| Cooperative | S03 | 48.9° C. | 52.2° C. | |
| Cooperative | S04 | 49.3° C. | 52.4° C. | |
| Standard | S05 | 63.1° C. | | |
| Standard | S06 | 71.6° C. | | |

The reaction mixture had a volume of 20 μL and included 0.2 mM of each deoxynucleotide triphosphate (Sigma-Aldrich, St. Louis, Missouri), 0.5 μM of each primer, 20 μM of Maverick Blue (Co-Diagnostics, Inc., Salt Lake City, Utah), 50 mM Tris pH 8.3, 0.125 mg/mL bovine serum albumin (Sigma), 3 mM MgCl2 (Sigma), 0.156 U/μL GoTaq MDx Hot Start Polymerase (Promega), with or without 0.1 U/μL qScript Reverse Transcriptase (QuantaBio, Beverly, MA), and with or without RNA equivalent of 0.3 μl of Tabasco sauce. Standard primers, cooperative primers, and CopTers were obtained from IDT (Coralvilee, Iowa), and were designed to amplify the same region of the PMMV genome. For each primer set, three separate reactions were performed: with RNA template, without RNA template, and without reverse transcriptase. Using the Cielo 6 Real Time qPCR system, (Azure Biosystems, Dublin, CA), all samples were incubated at 55° C. for 60 s (whether or not template or reverse transcriptase were included), then denatured at 95° C. for 60 s, followed by 30 to 60 amplification cycles of 95° C., 10 s denaturation, and 10 s annealing at varying temperatures.

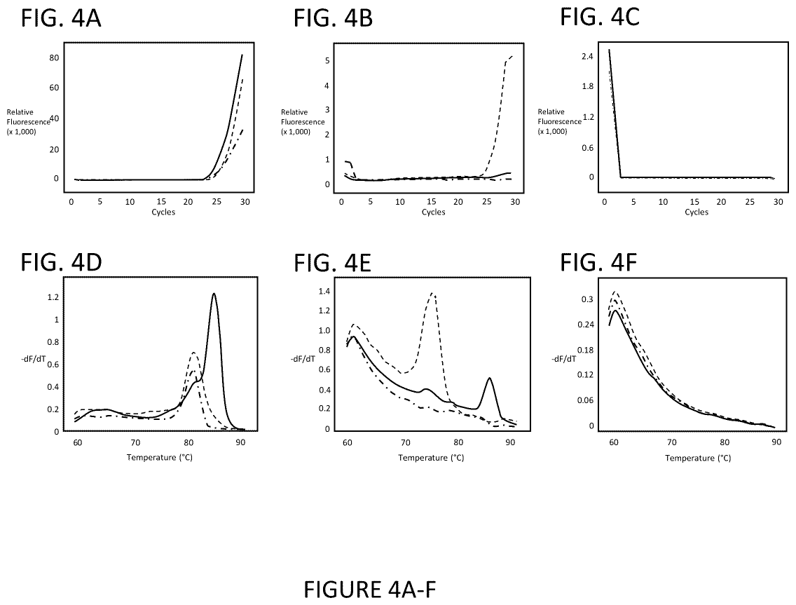

When annealing was performed at 55° C., standard and cooperative primer sets generated amplification products, as shown in FIGS. 4A and 4B, respectively. However, non-specific amplification and accumulation of primer dimers were observed as shown in FIGS. 4D and 4D where amplification products with melting temperatures lower than that of the expected PMMV amplicon (85° C.) were obvious, particularly in the absence of template or reverse transcriptase. In contrast, CopTers were unable to amplify anything at this annealing temperature as shown by the flat line in the amplification curve (FIG. 4C) and the lack of melting peak (FIG. 4F) (note the scale difference between FIGS. 4C and 4F versus 4A, 4B, and 4C, 4D respectively).

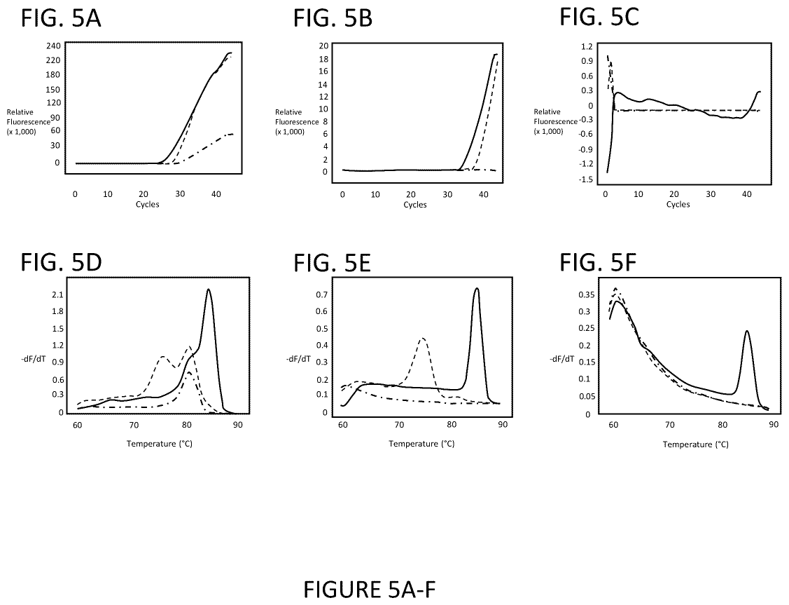

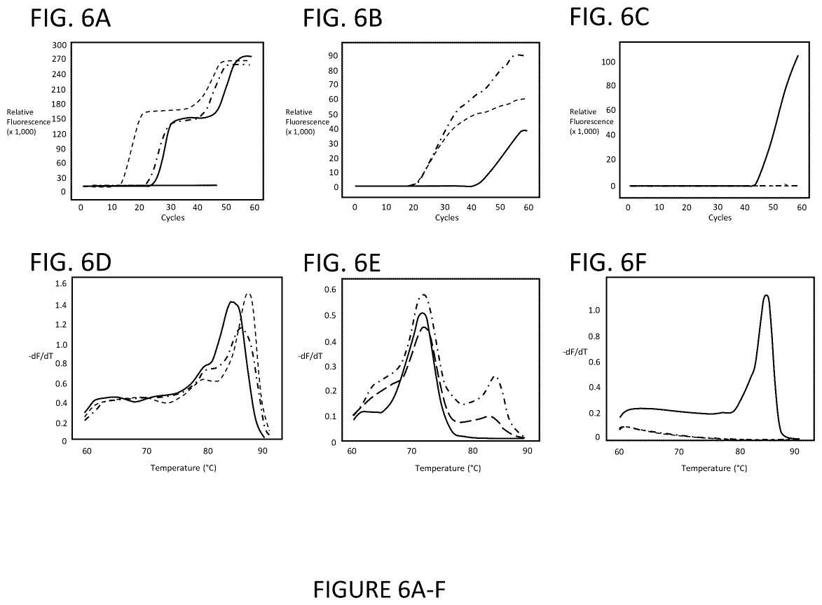

When the annealing temperature was raised to 60° C., standard primers (FIG. 5A, 5D) and cooperative primers (FIG. 5B, 5E) continued to show non-specific amplification and primer dimers, but CopTers produced a single amplification product at the expected melting temperature of the PMMV amplicon (FIG. 5F) despite weak amplification (FIG. 5C). Once the annealing temperature was raised to 67° C., CopTers generated robust specific amplification (FIG. 6C) of a single amplicon of PMMV (FIG. 6F). Reactions without RNA template or reverse transcriptase did not show amplification products. Standard primers (FIG. 6A, 6D) and cooperative primers (FIG. 6B, 6E) continued to generate non-specific products.

### Example 3: SRP19 DNA Assay

Pairs of standard primers and of CopTer primers were both designed to amplify the same region of the human DNA template (the SRP19 gene). Sequences of the primers are shown in Table 3.

TABLE 3

| Type of Primer | ID | Sequence (5′ to 3′) |
|---|---|---|
| CopTer | S07 | CGAGACAAGGATTTTGGG (SEQ ID NO: 21) |
| | | /Sp18/TTTAAGTATTGTTGCG (SEQ ID NO: 22) |
| | | /Sp18/CTCAGGGCTCC (SEQ ID NO: 23) |
| CopTer | S08 | CATGTCCACGTAGCAC (SEQ ID NO: 24) |
| | | /Sp18/CCCTATGGTCGGAATTTC (SEQ ID NO: 25) |
| | | /Sp18/AGCCTTTCTGAAC (SEQ ID NO: 26) |
| Standard | S09 | AGCAGCCACCAGGCTCT (SEQ ID NO: 27) |
| Standard | S10 | AACCTTAAAATTTCACAAATGAGCC (SEQ ID NO: 28) |

The estimated melting temperatures of each annealing sequences are shown in Table 4.

TABLE 4

| | | Estimated Melting Temperatures of | | |
|---|---|---|---|---|
| Type of | | Priming | Anchor 1 | Anchor 2 |
| Primer | ID | sequence | sequence | sequence |
| CopTer | S07 | 48.3° C. | 50.0° C. | 59.5° C. |
| CopTer | S08 | 48.0° C. | 58.6° C. | 58.6° C. |
| Standard | S09 | 67.8° C. | | |
| Standard | S10 | 62.4° C. | | |

PCR was performed using 50 mM Tris pH 8.3, 0.125 mg/mL BSA, and 3 mM MgCl₂, 0.2 mM dNTPs, 0.04 U/μL KlenTaq 1 (DNA Polymerase Technologies, St Louis, MO), 0.5 μM primers or CopTers, 20 μM Maverick Blue with or without 5 ng/μL of human genomic DNA. PCR was performed on the Light Scanner-32 (BioFire Defense, Salt Lake City, UT) using a 1-minute denaturation step at 95° C. followed by 44 cycles of 95° C. at 5 s and 67° C. for 15 s.

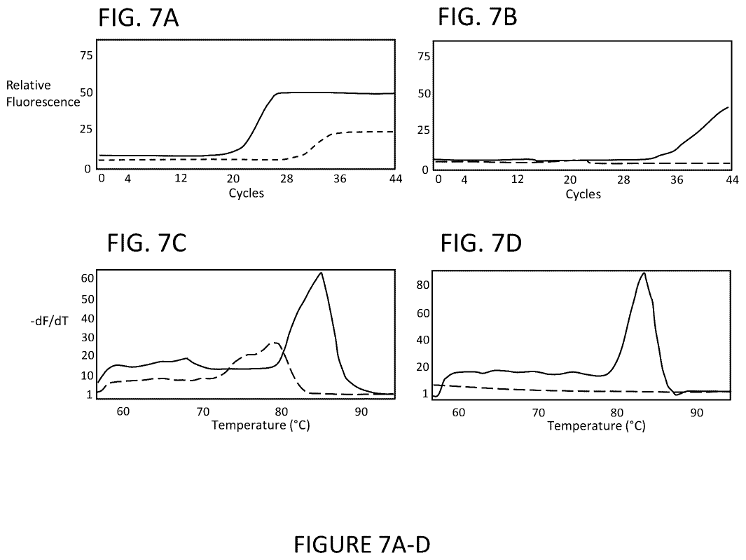

Using an annealing temperature of 67° C. during PCR, CopTers amplified a single product with a melting temperature of the expected SRP19 amplicon (FIG. 7B). No amplification was observed without the DNA template (FIG. 7B, 7D). In contrast, reactions using standard primers generated primer-dimers in the absence of DNA template (FIG. 7A, 7C).

### Example 4: SARS-CoV-2 RNA Assay

CopTer primer pairs were designed to amplify two conserved regions of the SARS-CoV-2 genome at locations 981nt and 12539nt (Wuhan genome coordinates). Each CopTer sequences are shown in Table 5.

TABLE 5

| Type of | | |
|---|---|---|
| Primer | ID | Sequence (5′ to 3′) |
| CopTer | S11 | AGAAATTTGACACCTTCAATGG (SEQ ID NO: 29) |
| 981 nt | | /iSp18/GATGGCTTTATGGGTAGAATTCGATCTGT (SEQ ID NO: 30) |
| | | /iSp18/CTTGGTACACGGAAC (SEQ ID NO: 31) |
| CopTer | S12 | GCTTTTTCTTTTCAACCCTTG (SEQ ID NO: 32) |
| 981 nt | | /iSp18/TTTCAAAAGGTGTCTGCAATTCATAGCTC (SEQ ID NO: 33) |
| | | /iSp18/TGACGCAACTGGATA (SEQ ID NO: 34) |
| CopTer | S13 | GTTGTAGATGCAGATAGTAAAATTGTT (SEQ ID NO: 35) |
| 12539 nt | | /iSp18/GCTTTAAGGGCCAATTCTGCTGTCA (SEQ ID NO: 36) |
| | | /iSp18/CCAGACTATAACACATATAAAA (SEQ ID NO: 37) |
| CopTer | S14 | TGTTACAATAAGAGGCCATGC (SEQ ID NO: 38) |
| 12539 nt | | /iSp18/CTGTTGGATTTCCCACAATGCTGATGCAT (SEQ ID NO: 39) |
| | | /iSp18/GGACTAAGCTCATTATTCT (SEQ ID NO: 40) |

The estimated melting temperatures of each annealing sequences are shown in Table 6.

TABLE 6

| | | Estimated Melting Temperatures of | | |
|---|---|---|---|---|
| Type of | | Priming | Anchor 1 | Anchor 2 |
| Primer | ID | sequence | sequence | sequence |
| CopTer 981 nt | S11 | 54.6° C. | 60.8° C. | 67.7° C. |
| CopTer 981 nt | S12 | 55.2° C. | 60.1° C. | 67.2° C. |
| CopTer 12539 nt | S13 | 54.4° C. | 61.5° C. | 69.2° C. |
| CopTer 12539 nt | S14 | 55.0° C. | 62.2° C. | 71.0° C. |

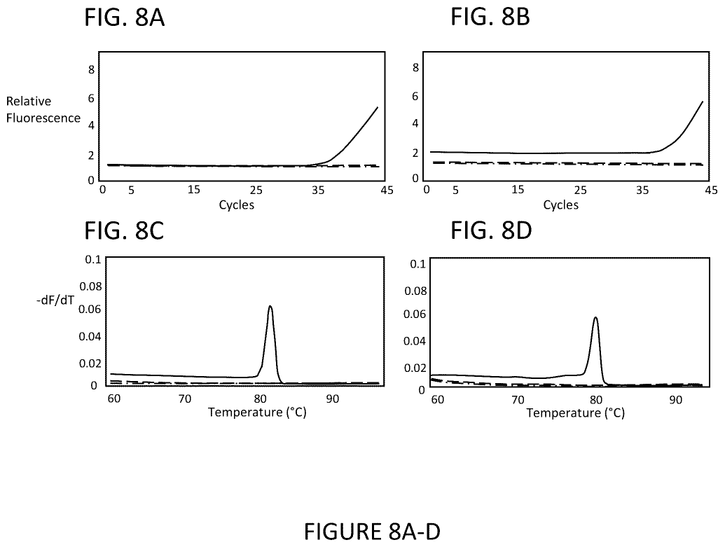

Template RNA was prepared from a 2 mL oral rinse specimen obtained from a patient who tested positive for SARS-CoV-2. The specimen was treated with 1 mg/mL of Proteinase K (LGC Biosearch Technologies, Novato, CA) at 60° C. for 5 minutes, and heat denatured at 95° C. for 5 minutes. Ten μL of this lysate was used in 20 μL reverse transcription PCR amplification mixtures which contained 50 mM Tris pH 8.3, 0.125 mg/mL BSA, and 4 mM MgCl₂, 0.2 mM dNTPs, 1× QScript, 0.04 U/μL KlenTaq, 0.5 μM CopTer primers, and 20 μM Maverick Blue. The reaction mixtures were incubated on the Magnetic Induction Cycler for reverse transcription at 55° C. for 5 minutes, then 95° C. for 5 minutes, followed by 45 cycles of PCR using the cycling conditions of 95° C. for 15 s, 67° C. for 20 s, and 72° C. for 15 s. The reaction that contained the CopTers targeting the 981 region amplified at a Cq of approximately 32 (FIG. 8A), while the CopTers targeting the 12539 region amplified at a Cq of 35 (FIG. 8B). No amplification was seen in either of the assays in the absence of template or reverse transcriptase, and there was no evidence of primer dimer formation in the melting curve plots for the 981 CopTers (FIG. 8C) or the 12539 CopTers (FIG. 8D).

It must be noted that as used herein and in the appended claims, the singular forms “a”, “an”, and “the” include plural reference unless the context clearly dictates otherwise. Thus, for example, reference to “a primer” includes a plurality of such primers, reference to “the primer” is a reference to one or more primers and equivalents thereof known to those skilled in the art, and so forth.

SEQUENCES

SEQ ID NO: 1
GGCAGGACCGTGAGAGAATTCC
SEQ ID NO: 2
AGTGATTACCGGCAAGAAGAACGCA
SEQ ID NO: 3
ATTTGCTTCAGGTGGTTGGCT
SEQ ID NO: 4
TGGAATATGAAGAACAGGGCTTC
SEQ ID NO: 5
CGGCAGGACCGUGAGAGAAUUCCAGCCAACCACCUGAAGCAAAUCUAGG
UAAGUCUAUGUGCGUUCUUCUUGCCGGUAAUCACUUCGCUGAAGCCCUG
UUCUUCAUAUUCCAU
SEQ ID NO: 6
CGGCAGGACCGTGAGAGAATTC
SEQ ID NO: 7
GACCTGATGGTCATCAGATGGAATATGAAGAACAGGGCTTCAGCGAAGT
GATTACCGGCAAGAAGAACGCACAGGGATTTGCTTCAGGTGGTTGGCTG
GAATTCTCTCACGGTCCTGCCGGTCCC
SEQ ID NO: 8
TTCAAATGAGAGTGGT
SEQ ID NO: 9
TGTCGCACTTGCATTGCA
SEQ ID NO: 10
GGCGTAGATCC
SEQ ID NO: 11
TTGCTTCGGTAGG
SEQ ID NO: 12
GGCTACCATTACCTTTGCTGC
SEQ ID NO: 13
GCAATTGTCGG
SEQ ID NO: 14
TGGTGGCAGCA
SEQ ID NO: 15
AAAAAA
SEQ ID NO: 16
GGCGTAGATCCA
SEQ ID NO: 17
ATGCAAGTGCGAC
SEQ ID NO: 18
AATTGTCGGTTGC
SEQ ID NO: 19
GCAGCAAAGGTAATGGTAGC
SEQ ID NO: 20
TGCAAGTGCGACATTTGCTTCGGT
SEQ ID NO: 21
CGAGACAAGGATTTTGGG
SEQ ID NO: 22
TTTAAGTATTGTTGCG
SEQ ID NO: 23
CTCAGGGCTCC
SEQ ID NO: 24
CATGTCCACGTAGCAC
SEQ ID NO: 25
CCCTATGGTCGGAATTTC
SEQ ID NO: 26
AGCCTTTCTGAAC
SEQ ID NO: 27
AGCAGCCACCAGGCTCT
SEQ ID NO: 28
AACCTTAAAATTTCACAAATGAGCC
SEQ ID NO: 29
AGAAATTTGACACCTTCAATGG
SEQ ID NO: 30
GATGGCTTTATGGGTAGAATTCGATCTGT
SEQ ID NO: 31
CTTGGTACACGGAAC
SEQ ID NO: 32
GCTTTTTCTTTTCAACCCTTG
SEQ ID NO: 33
TTTCAAAAGGTGTCTGCAATTCATAGCTC
SEQ ID NO: 34
TGACGCAACTGGATA
SEQ ID NO: 35
GTTGTAGATGCAGATAGTAAAATTGTT
SEQ ID NO: 36
GCTTTAAGGGCCAATTCTGCTGTCA
SEQ ID NO: 37
CCAGACTATAACACATATAAAA
SEQ ID NO: 38
TGTTACAATAAGAGGCCATGC
SEQ ID NO: 39
CTGTTGGATTTCCCACAATGCTGATGCAT
SEQ ID NO: 40
GGACTAAGCTCATTATTCT

We claim:

1. A nucleic acid molecule comprising:
a. a first extendable nucleic acid sequence configured to hybridize to a first region of a target nucleic acid;
b. a second nucleic acid sequence that comprises a non-extendable 3′ terminus, wherein said second nucleic acid sequence is located on a 5′ side of the first nucleic acid on the molecule and is configured to hybridize to a second region of the target nucleic acid downstream from a binding site of the first nucleic acid sequence relative to its direction of extension;
c. a first spacer connecting the first and second nucleic acid sequences configured to allow both sequences to hybridize to their targets, and comprising at least one non-nucleotide linker;
d. a third nucleic acid sequence that comprises a non-extendable 3′ terminus, wherein said third nucleic acid sequence is located on a 5′ side of the second nucleic acid on the molecule and is configured to hybridize to a third region of the target nucleic acid between where the 3′ end of the first nucleic acid sequence hybridizes and where the 5′ end of the second nucleic acid sequence hybridizes.

2. The nucleic acid molecule of claim 1, wherein the second and third nucleic acid sequences are connected by a second spacer.

3. The nucleic acid molecule of claim 1 or 2, wherein the molecule comprises at least one additional nucleic acid sequence which comprise at least one non-extendable moiety at it's 3′ terminus, wherein said non-extendable moiety renders said additional nucleic acid sequence non-extendable, wherein said additional nucleic acid sequence hybridizes to the target nucleic acid.

4. The nucleic acid molecule of any one of claims 1-3, wherein the first nucleic acid sequence cannot hybridize to its complementary target at a temperature that is at least 1° C. higher than its own melting temperature, without the second or third nucleic acid sequences also hybridizing to the target.

5. The nucleic acid molecule of claim 4, wherein the higher temperature is at least 8° C. higher.

6. The nucleic acid molecule of claim 5, wherein the higher temperature is between 8° C. and 15° C. higher.

7. The nucleic acid molecule of any one of claims 1-6, wherein the nucleic acid molecule comprises one or more nucleic acid modifications, but remains extendable at its 3′ end.

8. The nucleic acid molecule of any one of claims 1-7, wherein either the second, third, or both nucleic acid sequences comprise nucleic acid modifications which are not at the 3′ terminus.

9. The nucleic acid molecule of any one of claims 1-8, wherein the target nucleic acid is a single molecule.

10. The nucleic acid molecule of any one of claims 1-9, wherein at least one of the nucleic acid sequences comprises a detectable label.

11. The nucleic acid molecule of claim 10, wherein the detectable label is on the third nucleic acid sequence.

12. The nucleic acid molecule of claim 10 or 11, wherein the detectable label comprises a fluorescent resonance energy transfer (FRET) system.

13. The nucleic acid molecule of claim 10 or 11, wherein the detectable label comprises a cleavable moiety.

14. A method of nucleic acid amplification comprising the steps of:
a. providing a set of primers, wherein a first primer of the primer set comprises a tertiary cooperative primer comprising the nucleic acid molecule of claim 1;
b. exposing the set of primers of step a) to a target nucleic acid under conditions suitable for nucleic acid amplification, wherein at least part of the primers are complementary to at least part of the target nucleic acid; and
c. amplifying the target nucleic acid.

15. The method of claim 14, wherein the amplifying step comprises PCR.

16. The method of claim 15, wherein said PCR comprises reverse transcription PCR (RT-PCR).

17. The method of any one of claims 14-16, wherein the second primer is selected from a group consisting of a second tertiary cooperative primer, a standard (non-cooperative) primer, and a cooperative primer.

18. The method of claim 17, wherein the amplifying step is preceded by reverse transcription.

19. The method of claim 18, wherein the reverse transcription is performed at 60° C. to 70° C. by a thermostable reverse transcriptase.

20. The method of claim 14 or 15, wherein the amplifying step is performed by use of a thermostable DNA polymerase that lacks 5′→3′ exonuclease activity.

21. The method of claim 14 or 15, wherein the amplifying step comprises extension steps that occur between 72° C. and 80° C.

22. The method of claim 14 or 15, wherein the amplifying step comprises annealing steps that occur between 64° C. and 72° C.

23. The method of claim 14 or 15, wherein a single-stranded DNA binding protein is added during the step of amplification.

24. The method of any one of claims 14-23, wherein at least one of the primers is detectably labeled.

25. The method of claim 24, wherein the label is on a 5′ terminal anchor sequence of the first primer.

26. The method of claim 25, wherein the label is a cleavable moiety.

27. The method of claim 26, wherein the label produces a detectable signal when hydrolyzed by 5′ nuclease activity of a thermostable DNA polymerase used for PCR.

28. The method of claim 24, wherein the label comprises:
a. a double-stranded DNA binding dye in the reaction mixture that acts as a fluorescent donor, and
b. a label on the tertiary cooperative primer acts as a fluorescent acceptor; wherein amplification of the target sequence occurs by illuminating the reaction mixture at an excitation wavelength of the double-stranded DNA binding dye and detecting florescence at a wavelength of emission of the label.

29. The method of any one of claims 14-28, wherein the nucleic acid amplification step comprises or is followed by melting analysis.

30. The method of any one of claims 14-29, wherein the set of primers are provided in an amplification reaction mixture.

31. A kit for amplification, wherein the kit comprises:
a) a tertiary cooperative primer according to claim 1; and
b) a thermostable polymerase.

32. The kit of claim 31, wherein the kit further comprises additional primers.

33. The kit of claim 32, wherein at least one additional primer is another tertiary cooperative primer.

34. The kit of claim 33, wherein the additional primer comprises a cooperative primer.

35. The kit of claim 34, wherein the additional primer is a standard (non-cooperative) primer.

36. The kit of any one of claims 31-35, wherein the kit further comprises reagents useful for the amplification of nucleic acid.

37. The kit of claim 36, wherein reagents further comprise amplification buffer.

38. The kit of any one of claims 31-37, wherein the kit further comprises a double-stranded DNA binding dye.

39. The kit of any one of claims 31-38, wherein the tertiary cooperative primer is labeled with one or more fluorescent labels and optionally with one or more quenchers.

40. The kit of any one of claims 31-39, wherein one or more thermostable polymerases are selected from a group consisting of thermostable DNA polymerase and thermostable reverse transcriptase.

41. A reaction mixture comprising a tertiary cooperative primer according to claim 1; and a thermostable polymerase.


SEQUENCE LISTING (40 sequences)

SEQ ID NO: 1 - DNA, 22 bp, synthetic construct
  misc_feature 1..22: note = Description of sequence: Primer
  source 1..22: mol_type = other DNA
ggcaggaccgtgagagaattcc

SEQ ID NO: 2 - DNA, 25 bp, synthetic construct
  misc_feature 1..25: note = Description of sequence: Primer
  source 1..25: mol_type = other DNA
  misc_feature 25: note = Nucleotide bound to an 18-atom non-nucleotide hexa-ethyleneglycol spacer
agtgattaccggcaagaagaacgca

SEQ ID NO: 3 - DNA, 21 bp, synthetic construct
  misc_feature 1..21: note = Description of sequence: Primer
  source 1..21: mol_type = other DNA
  misc_feature 1: note = Nucleotide bound to an 18-atom non-nucleotide hexa-ethyleneglycol spacer
atttgcttcaggtggttggct

SEQ ID NO: 4 - DNA, 23 bp, synthetic construct
  misc_feature 1..23: note = Description of sequence: Primer
  source 1..23: mol_type = other DNA
tggaatatgaagaacagggcttc

SEQ ID NO: 5 - RNA, 113 nt, synthetic construct
  misc_feature 1..113: note = Description of sequence: synthetic RNA template
  source 1..113: mol_type = other RNA
  misc_binding 2..23: bound_moiety = Primer and CopTer binding sites
  misc_binding 90..112: bound_moiety = Primer and CopTer binding sites
cggcaggaccgtgagagaattccagccaaccacctgaagcaaatctaggtaagtctatgtgcgttcttcttgccggtaatcacttcgctgaagccctgttcttcatattccat

SEQ ID NO: 6 - DNA, 22 bp, synthetic construct
  misc_feature 1..22: note = Description of sequence: Primer
  source 1..22: mol_type = other DNA
cggcaggaccgtgagagaattc

SEQ ID NO: 7 - DNA, 125 bp, synthetic construct
  misc_feature 1..125: note = Description of sequence: synthetic RNA template
  source 1..125: mol_type = other DNA
  misc_binding 19..41: bound_moiety = Primer and CopTer binding sites
  misc_binding 99..120: bound_moiety = Primer and CopTer binding sites
gacctgatggtcatcagatggaatatgaagaacagggcttcagcgaagtgattaccggcaagaagaacgcacagggatttgcttcaggtggttggctggaattctctcacggtcctgccggtccc

SEQ ID NO: 8 - DNA, 16 bp, synthetic construct
  misc_feature 1..16: note = Description of sequence: Primer
  source 1..16: mol_type = other DNA
  misc_feature 16: note = Nucleotide bound to an 18-atom non-nucleotide hexa-ethyleneglycol spacer
ttcaaatgagagtggt

SEQ ID NO: 9 - DNA, 18 bp, synthetic construct
  misc_feature 1..18: note = Description of sequence: Primer
  source 1..18: mol_type = other DNA
  misc_feature 1: note = Nucleotide bound to an 18-atom non-nucleotide hexa-ethyleneglycol spacer
  misc_feature 18: note = Nucleotide bound to an 18-atom non-nucleotide hexa-ethyleneglycol spacer
tgtcgcacttgcattgca

SEQ ID NO: 10 - DNA, 11 bp, synthetic construct
  misc_feature 1..11: note = Description of sequence: Primer
  source 1..11: mol_type = other DNA
  misc_feature 1: note = Nucleotide bound to an 18-atom non-nucleotide hexa-ethyleneglycol spacer
ggcgtagatcc

SEQ ID NO: 11 - DNA, 13 bp, synthetic construct
  misc_feature 1..13: note = Description of sequence: Primer
  source 1..13: mol_type = other DNA
  misc_feature 13: note = Nucleotide bound to an 18-atom non-nucleotide hexa-ethyleneglycol spacer
ttgcttcggtagg

SEQ ID NO: 12 - DNA, 21 bp, synthetic construct
  misc_feature 1..21: note = Description of sequence: Primer
  source 1..21: mol_type = other DNA
  misc_feature 1: note = Nucleotide bound to an 18-atom non-nucleotide hexa-ethyleneglycol spacer
  misc_feature 21: note = Nucleotide bound to an 18-atom non-nucleotide hexa-ethyleneglycol spacer
ggctaccattacctttgctgc

SEQ ID NO: 13 - DNA, 11 bp, synthetic construct
  misc_feature 1..11: note = Description of sequence: Primer
  source 1..11: mol_type = other DNA
  misc_feature 1: note = Nucleotide bound to an 18-atom non-nucleotide hexa-ethyleneglycol spacer
gcaattgtcgg

SEQ ID NO: 14 - DNA, 11 bp, synthetic construct
  misc_feature 1..11: note = Description of sequence: Primer
  source 1..11: mol_type = other DNA
  misc_feature 11: note = Nucleotide bound to an 18-atom non-nucleotide hexa-ethyleneglycol spacer
tggtggcagca

SEQ ID NO: 15 - skipped

SEQ ID NO: 16 - DNA, 12 bp, synthetic construct
  misc_feature 1..12: note = Description of sequence: Primer
  source 1..12: mol_type = other DNA
  misc_feature 1: note = Nucleotide bound to an 18-atom non-nucleotide hexa-ethyleneglycol spacer
ggcgtagatcca

SEQ ID NO: 17 - DNA, 13 bp, synthetic construct
  misc_feature 1..13: note = Description of sequence: Primer
  source 1..13: mol_type = other DNA
  misc_feature 13: note = Nucleotide bound to an 18-atom non-nucleotide hexa-ethyleneglycol spacer
atgcaagtgcgac

SEQ ID NO: 18 - DNA, 13 bp, synthetic construct
  misc_feature 1..13: note = Description of sequence: Primer
  source 1..13: mol_type = other DNA
  misc_feature 1: note = Nucleotide bound to an 18-atom non-nucleotide hexa-ethyleneglycol spacer
aattgtcggttgc

SEQ ID NO: 19 - DNA, 20 bp, synthetic construct
  misc_feature 1..20: note = Description of sequence: Primer
  source 1..20: mol_type = other DNA
gcagcaaaggtaatggtagc

SEQ ID NO: 20 - DNA, 24 bp, synthetic construct
  misc_feature 1..24: note = Description of sequence: Primer
  source 1..24: mol_type = other DNA
tgcaagtgcgacatttgcttcggt

SEQ ID NO: 21 - DNA, 18 bp, synthetic construct
  misc_feature 1..18: note = Description of sequence: Primer
  source 1..18: mol_type = other DNA
  misc_feature 18: note = Nucleotide bound to an 18-atom non-nucleotide hexa-ethyleneglycol spacer
cgagacaaggattttggg

SEQ ID NO: 22 - DNA, 16 bp, synthetic construct
  misc_feature 1..16: note = Description of sequence: Primer
  source 1..16: mol_type = other DNA
  misc_feature 1: note = Nucleotide bound to an 18-atom non-nucleotide hexa-ethyleneglycol spacer
  misc_feature 16: note = Nucleotide bound to an 18-atom non-nucleotide hexa-ethyleneglycol spacer
tttaagtattgttgcg

SEQ ID NO: 23 - DNA, 11 bp, synthetic construct
  misc_feature 1..11: note = Description of sequence: Primer
  source 1..11: mol_type = other DNA
  misc_feature 1: note = Nucleotide bound to an 18-atom non-nucleotide hexa-ethyleneglycol spacer
ctcagggctcc

SEQ ID NO: 24 - DNA, 16 bp, synthetic construct
  misc_feature 1..16: note = Description of sequence: Primer
  source 1..16: mol_type = other DNA
  misc_feature 16: note = Nucleotide bound to an 18-atom non-nucleotide hexa-ethyleneglycol spacer
catgtccacgtagcac

SEQ ID NO: 25 - DNA, 18 bp, synthetic construct
  misc_feature 1..18: note = Description of sequence: Primer
  source 1..18: mol_type = other DNA
  misc_feature 1: note = Nucleotide bound to an 18-atom non-nucleotide hexa-ethyleneglycol spacer
  misc_feature 18: note = Nucleotide bound to an 18-atom non-nucleotide hexa-ethyleneglycol spacer
ccctatggtcggaatttc

SEQ ID NO: 26 - DNA, 13 bp, synthetic construct
  misc_feature 1..13: note = Description of sequence: Primer
  source 1..13: mol_type = other DNA
  misc_feature 1: note = Nucleotide bound to an 18-atom non-nucleotide hexa-ethyleneglycol spacer
agcctttctgaac

SEQ ID NO: 27 - DNA, 17 bp, synthetic construct
  misc_feature 1..17: note = Description of sequence: Primer
  source 1..17: mol_type = other DNA
agcagccaccaggctct

SEQ ID NO: 28 - DNA, 25 bp, synthetic construct
  misc_feature 1..25: note = Description of sequence: Primer
  source 1..25: mol_type = other DNA
aaccttaaaatttcacaaatgagcc

SEQ ID NO: 29 - DNA, 22 bp, synthetic construct
  misc_feature 1..22: note = Description of sequence: Primer
  source 1..22: mol_type = other DNA
  misc_feature 22: note = Nucleotide bound to an 18-atom non-nucleotide hexa-ethyleneglycol spacer
agaaatttgacaccttcaatgg

SEQ ID NO: 30 - DNA, 29 bp, synthetic construct
  misc_feature 1..29: note = Description of sequence: Primer
  source 1..29: mol_type = other DNA
  misc_feature 1: note = Nucleotide bound to an 18-atom non-nucleotide hexa-ethyleneglycol spacer
  misc_feature 29: note = Nucleotide bound to an 18-atom non-nucleotide hexa-ethyleneglycol spacer
gatggctttatgggtagaattcgatctgt

SEQ ID NO: 31 - DNA, 15 bp, synthetic construct
  misc_feature 1..15: note = Description of sequence: Primer
  source 1..15: mol_type = other DNA
  misc_feature 1: note = Nucleotide bound to an 18-atom non-nucleotide hexa-ethyleneglycol spacer
cttggtacacggaac

SEQ ID NO: 32 - DNA, 21 bp, synthetic construct
  misc_feature 1..21: note = Description of sequence: Primer
  source 1..21: mol_type = other DNA
  misc_feature 21: note = Nucleotide bound to an 18-atom non-nucleotide hexa-ethyleneglycol spacer
gctttttcttttcaacccttg

SEQ ID NO: 33 - DNA, 29 bp, synthetic construct
  misc_feature 1..29: note = Description of sequence: Primer
  source 1..29: mol_type = other DNA
  misc_feature 1: note = Nucleotide bound to an 18-atom non-nucleotide hexa-ethyleneglycol spacer
  misc_feature 29: note = Nucleotide bound to an 18-atom non-nucleotide hexa-ethyleneglycol spacer
tttcaaaaggtgtctgcaattcatagctc

SEQ ID NO: 34 - DNA, 15 bp, synthetic construct
  misc_feature 1..15: note = Description of sequence: Primer
  source 1..15: mol_type = other DNA
  misc_feature 1: note = Nucleotide bound to an 18-atom non-nucleotide hexa-ethyleneglycol spacer
tgacgcaactggata

SEQ ID NO: 35 - DNA, 27 bp, synthetic construct
  misc_feature 1..27: note = Description of sequence: Primer
  source 1..27: mol_type = other DNA
  misc_feature 27: note = Nucleotide bound to an 18-atom non-nucleotide hexa-ethyleneglycol spacer
gttgtagatgcagatagtaaaattgtt

SEQ ID NO: 36 - DNA, 25 bp, synthetic construct
  misc_feature 1..25: note = Description of sequence: Primer
  source 1..25: mol_type = other DNA
  misc_feature 1: note = Nucleotide bound to an 18-atom non-nucleotide hexa-ethyleneglycol spacer
  misc_feature 25: note = Nucleotide bound to an 18-atom non-nucleotide hexa-ethyleneglycol spacer
gctttaagggccaattctgctgtca

SEQ ID NO: 37 - DNA, 22 bp, synthetic construct
  misc_feature 1..22: note = Description of sequence: Primer
  source 1..22: mol_type = other DNA
  misc_feature 1: note = Nucleotide bound to an 18-atom non-nucleotide hexa-ethyleneglycol spacer
ccagactataacacatataaaa

SEQ ID NO: 38 - DNA, 21 bp, synthetic construct
  misc_feature 1..21: note = Description of sequence: Primer
  source 1..21: mol_type = other DNA
  misc_feature 21: note = Nucleotide bound to an 18-atom non-nucleotide hexa-ethyleneglycol spacer
tgttacaataagaggccatgc

SEQ ID NO: 39 - DNA, 29 bp, synthetic construct
  misc_feature 1..29: note = Description of sequence: Primer
  source 1..29: mol_type = other DNA
  misc_feature 1: note = Nucleotide bound to an 18-atom non-nucleotide hexa-ethyleneglycol spacer
  misc_feature 29: note = Nucleotide bound to an 18-atom non-nucleotide hexa-ethyleneglycol spacer
ctgttggatttcccacaatgctgatgcat

SEQ ID NO: 40 - DNA, 19 bp, synthetic construct
  misc_feature 1..19: note = Description of sequence: Primer
  source 1..19: mol_type = other DNA
  misc_feature 1: note = Nucleotide bound to an 18-atom non-nucleotide hexa-ethyleneglycol spacer
ggactaagctcattattct